(19) United States
(12) Patent Application Publication
(10) Pub. No.: US 2019/0175956 A1
(43) Pub. Date: Jun. 13, 2019

(54) ORAL CARE PRODUCTS AND FORMULATIONS

(71) Applicant: David Dolezal, Scottsdale, AZ (US)

(72) Inventor: David Dolezal, Scottsdale, AZ (US)

(21) Appl. No.: 16/040,088

(22) Filed: Jul. 19, 2018

Related U.S. Application Data
(60) Provisional application No. 62/534,418, filed on Jul. 19, 2017.

(51) Int. Cl. A61Q 11/00; A61K 8/9789; A61K 8/9794; A61K 8/92; A61K 8/67; A61K 8/99; A61P 1/02; A61C 15/02

(52) U.S. Cl. CPC A61Q 11/00; A61K 8/9789; A61K 8/9794; A61C 15/02; A61K 8/67; A61K 8/99; A61P 1/02; A61K 8/922

(57) ABSTRACT

Oral care products, particularly safer, natural or more naturally derived, and/or less toxic or non-toxic oral care products and other consumer products. In particular, oral care products may include toothpastes, mouthwashes, whitening products, whitening wands, and oral care crystals comprising non-toxic and natural ingredients. In some embodiments, oral care products may include one or more essential oils. Oil-based and water-based components may be mixed using a homogenization process.

### CROSS-REFERENCE TO RELATED APPLICATIONS

This application relates to U.S. Provisional Patent Application No. 62/534,418, entitled Formulations for Oral Care Products and Other Formulations, and filed Jul. 19, 2017, the content of which is hereby incorporated by reference herein in its entirety.

### FIELD OF THE INVENTION

The present disclosure relates to oral care products. Particularly, the present disclosure relates to safer, natural or more naturally derived, and/or less toxic or non-toxic oral care products. Additionally, the present disclosure relates to oral care products that are customized to a user or experience.

### BACKGROUND OF THE INVENTION

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Traditional oral care products use a number of useless and often toxic ingredients. Traditional toothpastes, for example, may contain toxic ingredients such as sodium lauryl sulfate, triclosan, and/or sodium fluoride, which pose potential health risks and side effects, and may even require explicit warnings to contact poison control if swallowed. Consumer demand and ongoing pressure from many retailers have started a movement away from harmful chemicals and now the focus is on safer, environmentally friendly, and sustainable products. While labels such as “pure,” “natural,” and “organic” exist, there is limited legal standard required to use these labels and thus they are often meaningless. In addition, traditional oral care products are produced for the masses, despite the unique differences in people's biological makeups, habits, behaviors, styles, and preferences.

Thus, there is a need in the art for safer, natural or more naturally derived, and/or less toxic or non-toxic oral care products. Additionally, there is a need for healthy products that are free from chemicals, toxins, and artificial flavors and colors that are thought to be harmful.

### BRIEF SUMMARY OF THE INVENTION

The following presents a simplified summary of one or more embodiments of the present disclosure in order to provide a basic understanding of such embodiments. This summary is not an extensive overview of all contemplated embodiments and is intended to neither identify key or critical elements of all embodiments, nor delineate the scope of any or all embodiments.

The present disclosure, in one or more embodiments, relate to a toothpaste having purified water, xylitol, calcium citrate, and at least one essential oil. In some embodiments, the toothpaste may additionally have diatomaceous earth, malic acid, xanthan gum, arrowroot flour, and/or coconut oil. The toothpaste may have potassium sorbate, at least one vitamin, and/or at least one mineral. Essential oils may include such oils as: agar, ajwain, allspice, ambrette seed, amyris, angelica root, anise, arnica, asafetida, balsam of Peru, basil, holy basil, bay, bay laurel, beeswax, benzoin, bergamot, blue cypress, bois-de-rose, boronia, black pepper, buchu oil, birch, bitter almond, boldo, cajuput, camphor, brown camphor, yellow camphor, cannabis flower, caraway, cardamom seed, zingiberaceae, carrot seed, cedarwood, chamomile, chocolate (cacao), calamus root, cassia, cinnamon, citronella, clary sage, clove, clover, coffee, coriander, costmary, costus root, cumin, curry leaf, cypress, davana, deertongue, dill, elecampane, elemi, eucalyptus, fennel seed, fenugreek, fir, frankincense, galangal, galbanum, garlic, geranium, ginger, goldenrod, grapefruit, henna, helichrysum, hickory nut, horseradish, hyssop, Idaho tansy, jaborandi, jasmine, juniper berry, laurus nobilis, lavandin, lavender, ledum, lemon, lemon myrtle, lemongrass, lime, litsea cubeba, linaloe, mandarin, manuka, marjoram, melaleuca alternifolia, melilotus, Melissa oil, menthe arvensis, mint oil, mountain savory, mugwort, mullein, mustard, myrrh, myrtle, neem, neroli, niaouli, nutmeg, oakmoss, onion, sweet orange, bitter orange, oregano, orange, orris, palmarosa, Palo Santo, parsley, patchaouli, peppermint, perilla, pennyroyal, petitgrain, pimento, pine, ravensara, red cedar, roman chamomile, rose, rosehip, rosemary, rosewood, rue, sage, sandalwood, sassafras, savory, schisandra, Spanish broom, spearmint, spikenard, spruce, star anise, sweet birch, tagetes, tangerine, tansy, tarragon, tea tree, thuja, thyme, tobacco, tsuga, tuberose, turmeric, valerian, vanilla, violet leaf, Virginian, vetiver, western red cedar, wintergreen, wormseed, wormwood, yarrow, ylang-ylang, zedoary, and yuzu oil. In some embodiments, the toothpaste may include a blend of any of lemon oil, lime oil, sweet orange oil, ginger oil, tea tree oil, wintergreen oil, spearmint oil, peppermint oil, ylang ylang oil, vanilla oil, cinnamon oil, clove oil, grapefruit oil, eucalyptus oil, and myrrh oil. Components of the toothpaste may be combined using a homogenization process. Moreover, calcium in the toothpaste may be provided as calcium citrate, and xylitol may be provided as Birch Tree xylitol. In some embodiments, the toothpaste may have between about 20% and about 35% by volume of xylitol. In some embodiments, the toothpaste may have between about 20% and about 35% by volume of xylitol, between about 3% and about 15% by volume of calcium citrate, and between about 0.01% and about 4% by volume of at least one essential oil. In some embodiments, the toothpaste may have a pH of between 6.7 and 7.3.

The present disclosure, in one or more embodiments, additionally relates to an alcohol-free mouthwash having purified water, xylitol, and one or more essential oils. In some embodiments, the mouthwash may additionally have potassium sorbate, at least one vitamin, and at least one mineral. Essential oils may include such oils as: agar, ajwain, allspice, ambrette seed, amyris, angelica root, anise, arnica, asafetida, balsam of Peru, basil, holy basil, bay, bay laurel, beeswax, benzoin, bergamot, blue cypress, bois-de-rose, boronia, black pepper, buchu oil, birch, bitter almond, boldo, cajuput, camphor, brown camphor, yellow camphor, cannabis flower, caraway, cardamom seed, zingiberaceae, carrot seed, cedarwood, chamomile, chocolate (cacao), calamus root, cassia, cinnamon, citronella, clary sage, clove, clover, coffee, coriander, costmary, costus root, cumin, curry leaf, cypress, davana, deertongue, dill, elecampane, elemi, eucalyptus, fennel seed, fenugreek, fir, frankincense, galangal, galbanum, garlic, geranium, ginger, goldenrod, grapefruit, henna, helichrysum, hickory nut, horseradish, hyssop, Idaho tansy, jaborandi, jasmine, juniper berry, laurus nobilis, lavandin, lavender, ledum, lemon, lemon myrtle, lemongrass, lime, litsea cubeba, linaloe, mandarin, manuka, marjoram, melaleuca alternifolia, melilotus, Melissa oil, menthe arvensis, mint oil, mountain savory, mugwort, mullein, mustard, myrrh, myrtle, neem, neroli, niaouli, nutmeg, oakmoss, onion, sweet orange, bitter orange, oregano, orange, orris, palmarosa, Palo Santo, parsley, patchaouli, peppermint, perilla, pennyroyal, petitgrain, pimento, pine, ravensara, red cedar, roman chamomile, rose, rosehip, rosemary, rosewood, rue, sage, sandalwood, sassafras, savory, schisandra, Spanish broom, spearmint, spikenard, spruce, star anise, sweet birch, tagetes, tangerine, tansy, tarragon, tea tree, thuja, thyme, tobacco, tsuga, tuberose, turmeric, valerian, vanilla, violet leaf, Virginian, vetiver, western red cedar, wintergreen, wormseed, wormwood, yarrow, ylang-ylang, zedoary, and yuzu oil. In some embodiments, the mouthwash may include an essential oil blend of any of: lemon oil, lime oil, sweet orange oil, wintergreen oil, spearmint oil, peppermint oil, ylang ylang oil, vanilla oil, cinnamon oil, clove oil, grapefruit oil, eucalyptus oil, and myrrh oil. Components of the mouthwash may be combined using a homogenization process. In some embodiments, the mouthwash may include between about 7% and about 30% by volume of xylitol, and between about 0.02% and about 3.5% by volume of at least one essential oil. In some embodiments, the mouthwash may have a pH of between 6.7 and 7.3.

The present disclosure, in one or more embodiments, additionally relates to a whitening wand for removing or lightening stains and/or plaque from teeth. The whitening wand may have a handle and a porous whitening tip coupled to the handle. In some embodiments, the whitening tip may include a micro-compressed nano-technology sponge. In other embodiments, the whitening tip may include a wood or wood-based component. In some embodiments, the whitening tip may be readily removable from the handle.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter that is regarded as forming the various embodiments of the present disclosure, it is believed that the invention will be better understood from the following description taken in conjunction with the accompanying Figures, in which:

FIG. 1 shows a front view and profile view of a whitening wand of the present disclosure according to one embodiment.

FIG. 2 shows a front view and profile view of a whitening wand of the present disclosure according to one embodiment.

FIG. 3 is a profile view with cross sectional illustrations of a handle of a whitening wand of the present disclosure according to one embodiment.

FIG. 4 shows a back, profile, and top view of a whitening wand of the present disclosure according to one embodiment.

FIG. 5 shows a back, profile, and top view of a whitening wand of the present disclosure according to one embodiment.

FIG. 6 shows two cross sectional views of a whitening wand of the present disclosure according to one embodiment.

FIG. 7 shows an exploded back and profile view of a whitening wand of the present disclosure according to one embodiment.

FIG. 8A shows multiple views of a whitening tip and tip support of a whitening wand of the present disclosure according to one embodiment.

FIG. 8B shows multiple views of a whitening tip and tip support of a whitening wand of the present disclosure according to one embodiment.

FIG. 9A shows multiple views of a whitening tip of a whitening wand of the present disclosure according to one embodiment.

FIG. 9B shows multiple views of a whitening tip of a whitening wand of the present disclosure according to one embodiment.

FIG. 10A shows multiple views of a tip support of a whitening wand of the present disclosure according to one embodiment.

FIG. 10B shows multiple views of a tip support of a whitening wand of the present disclosure according to one embodiment.

FIG. 11 shows a back, profile, and top view of a cap of a whitening wand of the present disclosure according to one embodiment.

FIG. 12 shows two examples of packaging for a whitening wand of the present disclosure according to one embodiment.

FIG. 13 shows two profile views of a whitening wand of the present disclosure according to one embodiment.

FIG. 14 shows a portion of a whitening wand of the present disclosure according to one embodiment.

FIG. 15 shows a package of whitening tips and tip supports of the present disclosure according to one embodiment.

FIG. 16 shows a profile view of a whitening wand and a bottom view of a package for whitening tips of the present disclosure according to one embodiment.

FIG. 17 shows an exemplary pharmaceutical blister package.

FIG. 18 shows a perspective side view of a few whitening wands of the present disclosure according to another embodiment.

FIG. 19 shows a side view of a whitening wand of the present disclosure according to one embodiment.

FIG. 20 shows a side view of a dental brush of the present disclosure according to one embodiment.

FIG. 21 is a flow chart of the method to mix embodiments of the present disclosure.

### DETAILED DESCRIPTION

The present disclosure relates to novel and advantageous oral care products. Particularly, the present disclosure relates to novel and advantageous oral care products that are less toxic or non-toxic, sustainable, and generally safer than traditional oral care products. In various embodiments, products of the present disclosure may be toxin-free. In various embodiments, products of the present disclosure may be free of harmful chemicals. In still further embodiments, products of the present disclosure may be free of artificial flavors and/or artificial colors.

A particularly unique and advantageous aspect of the present disclosure is the combination of less toxic or non-toxic and natural or natural derivative components and ingredients to create products for oral care that may be adapted to individual users or experiences. For example, products may be tailored toward different tastes, such as a product directed toward strong, intense, and bold tastes, and a product directed toward gentle tingly, tantalizing, and intriguing tastes. Likewise, products according to the various embodiments of the present disclosure may be tailored to off-set, accommodate, or compensate for behavioral findings or preferences. For example, products may be tailored to encourage better dental or oral care. Products may be tailored toward addressing different issues or goals. For example, some products may be tailored toward tooth whitening, gum health, mouth sores, dry mouth, sensitive teeth, and/or other goals.

In various embodiments of the present disclosure, one or more products may be created to provide the requisite oral care but that are also tailored to the unique biological make-up, preferences, and/or behavioral activities of the user. One or more unique products may be used in order to accomplish this task, including but not limited to, toothpaste, mouthwash, floss, whitening paste or gel, denture cleaner, gum, whitening wands, and professional teeth polishers and cleaners. Products of the present disclosure may generally include natural or naturally-derived ingredients. A variety of natural or naturally-derived ingredients may provide various benefits to oral health. Antimicrobial ingredients may be configured to target oral pathogens, such as streptococcus mutans, while anti-inflammatory and wound healing ingredients may offer support to healthy gums and teeth. Other natural or naturally-derived ingredients may penetrate or help eliminate biofilm, thus in turn helping to mitigate plaque, tooth decay, gum disease, and the spread of bacteria from tooth roots to other areas of the body. An additional advantage of natural ingredients may be multifunctionality. Some ingredients may provide anti-inflammatory, immunological, and/or wound healing support, for example. The presence of secondary metabolites, such as phenolics in essential oils and tannins in herbal extracts may provide antimicrobial properties in some embodiments.

Essential Oils

In various embodiments of the present disclosure, oral care products may contain one or more essential oils. Essential oils may generally be from natural sources, such as plants. Essential oils may have an odor or taste and thus be used for perfumery or flavoring. By contrast, various essential oils may have deodorizing properties and thus be used to help prevent or reduce bad breath. By using one essential oil, or combining one or more essential oils, various flavor combinations may be formed.

Some essential oils, and other natural botanicals, may provide antimicrobial, antiseptic, and/or antibacterial benefits or properties to the product(s), making them effective for maintaining a healthy oral care routine. For example, some essential oils may help protect the mouth from acid-forming bacteria, whose presence may lead to periodontal disease. Some essential oils may penetrate the dentin and provide nutrition and/or other benefits to the entire tooth, including the roots.

Additionally, various essential oils have been shown to provide various health benefits, including but not limited to, acting as a pain reliever, digestive aid, diuretic, anti-ager, fever reducer, decongestant, memory booster, anti-inflammatory, antiviral, disinfectant, aphrodisiac, etc. Other beneficial health characteristics such as tightening gums and muscles as well as strengthening gums have also been shown. Moreover, various essential oils have been shown to be a relaxant and/or a stimulant. In addition, the beneficial effects derived from the essential oils may continue after use of the product(s).

In some embodiments, essential oils may be produced by stem hyper distillation to separate the oil components from other plant components. However, other distillation, extraction, separation, or production processes may be used.

Some of the essential oils that may be used in an oral care product formulation of the present disclosure include, but are not limited to:
The essential oil cinnamon, or cinnamon oil. Cinnamon has been shown to be an antibacterial. For example, cinnamon has been shown to be effective against streptococcus mutans, the bacteria responsible for tooth decay, as well as lactobacillus plantarum, one of the bacteria responsible in gum disease. Cinnamon has also been shown to have antifungal, antimicrobial, analgesic, anti-inflammatory, and antiseptic properties, among others. Cinnamon may also help to break down biofilm.
The essential oil clove, or clove oil. Clove has also been shown to have antibacterial properties, inhibiting many pathogenic bacteria such as Staph aureus, strep pneumonia, and shigella. Clove has also been shown to act as an antimicrobial, antiseptic, antiviral, natural fungicide, powerful disinfectant, germicide, and local anesthetic. Clove may also help to relieve pain, such as tooth pain. In addition, clove has been shown to be effective in combating bad breath and cavities. Clove may be particularly effective in inhibiting the growth of mutant and wild strains of bacteria.
The essential oil grapefruit, or grapefruit oil. Grapefruit may contain the powerful antioxidant d-limonene. Grapefruit has also been shown to act as an antiseptic, disinfectant, antibacterial, and stimulant. Grapefruit may also aid in detoxification.
The essential oil lemon, or lemon oil. Lemon may be a natural teeth whitener. Lemon may also enhance gum tissue. In addition, lemon has been shown to help fight infections, act as a detoxifier, and work as an antiseptic, disinfectant, antifungal, and astringent. Lemon may also be a significant source of Vitamin C, which may provide various benefits throughout the oral cavity and may help calcium and saliva to remineralize teeth.
The essential oil lime, or lime oil. Similar to lemon, lime may be a natural teeth whitener and help enhance or restore gum tissue. Likewise, lime has been shown to help fight infections, act as a detoxifier, and work as an antiseptic, disinfectant, antifungal, and astringent.
The essential oil orange, or orange oil. Orange oil may contain an active compound called limonene. Limonene may help support circulation, help heal skin, and boost vitamin C absorption. Orange oil has also been shown to inhibit the grown of MRSA. In addition, orange may also act an anti-inflammatory and antiseptic as well as promoting collagen and healing.
The essential oil peppermint, or peppermint oil. Peppermint has been shown to help fight mouth infections, gum disease, and bad breath. Peppermint has also been shown to act as an analgesic, anesthetic, antibacterial, anti-inflammatory, antifungal, antimicrobial, antiseptic, and astringent. In particular, peppermint has been shown to be effective at killing anaerobic bacteria, which thrive in the low oxygen environments below the gum line and can cause gum disease.
The essential oil spearmint, or spearmint oil. Spearmint may be soothing to gum tissue as well as enhance healing ability. Similar to other essential oils, spearmint has been shown to act as an analgesic, anesthetic, antibacterial, anti-inflammatory, antiseptic, and astringent. Spearmint may eliminate germs and help wounds heal more quickly.
The essential oil tea tree, or tea tree oil. Tea tree has been shown to work as a dental painkiller. Also, tea tree has been shown to act as an anti-inflammatory and antimicrobial, killing microorganisms associated with tooth decay and gum disease.
The essential oil vanilla, or vanilla oil. Vanilla has been shown to act as an antioxidant, analgesic, and anti-inflammatory.
The essential oil neem, or neem oil. Neem oil may be produced from the neem tree, also known as the margosa tree. Thus, neem may also be referred to as margosa, or margosa oil. Neem has been shown to act as an antiseptic and an astringent. In various embodiments, neem may work as a stain remover. In some embodiments, neem may also be used to decrease bad breath.
The essential oil pine, or pine oil, or a gum-based turpentine. Pine has been shown to act as a natural deodorizer, antibacterial, antiseptic, antifungal and disinfectant.
The essential oil myrrh, or myrrh oil. Myrrh may act as an antiseptic and may additionally help to sooth gums. Myrrh may be used to support gum health and to heal mouth ulcers. Myrrh may help gum tissue health by supporting the flow of blood to the tissue.
Additional or alternative essential oils may include, but are not limited to, agar, ajwain, allspice, ambrette seed, amyris, angelica root, anise, arnica, asafetida, balsam of Peru, basil, holy basil, bay, bay laurel, beeswax, benzoin, bergamot, blue cypress, bois-de-rose, boronia, black pepper, buchu oil, birch, bitter almond, boldo, cajuput, camphor, brown camphor, yellow camphor, cannabis flower, caraway, cardamom seed, zingiberaceae, carrot seed, cedarwood, chamomile, chocolate (cacao), calamus root, cassia, citronella, clary sage, clover, coffee, coriander, costmary, costus root, cumin, curry leaf, cypress, davana, deertongue, dill, elecampane, elemi, eucalyptus, fennel seed, fenugreek, fir, frankincense, galangal, galbanum, garlic, geranium, ginger, goldenrod, green tea, henna, helichrysum, hickory nut, horseradish, hyssop, Idaho tansy, jaborandi, jasmine, juniper berry, laurus nobilis, lavandin, lavender, ledum, lemon, lemongrass, lemon myrtle, litsea cubeba, linaloe, mandarin, manuka, marjoram, melaleuca alternifolia, melilotus, Melissa oil, menthe arvensis, mint oil, mountain savory, mugwort, mullein, mustard, myrtle, neroli, niaouli, nutmeg, oakmoss, onion, sweet orange, bitter orange, oregano, orris, palmarosa, Palo Santo, parsley, patchouli, perilla, pennyroyal, pepper, peppermint, petitgrain, pimento, pine, ravensara, red cedar, roman chamomile, rose, rosehip, rosemary, rosewood, rue, sage, sandalwood, sassafras, savory, schisandra, Spanish broom, spikenard, spruce, star anise, sweet birch, tagetes, tangerine, tansy, tarragon, thuja, thyme, tobacco, tsuga, tuberose, turmeric, valerian, violet leaf, Virginian, vetiver, western red cedar, wintergreen, wormseed, wormwood, yarrow, ylang-ylang, zedoary, yuzu, any other suitable essential oil, or any combination thereof.

Other ingredients may additionally or alternatively be used in oral care product formulations of the present disclosure. In various embodiments, ingredients may additionally or alternatively include, but are not limited to: zinc, potassium, vitamin D such as vitamin D3, vitamin K such as Vitamin K2, calcium such as but not limited to calcium citrate, calcium such as coral calcium derived from coral reef deposits, vitamin B, magnesium, sodium bicarbonate, xylitol, xanthum, coconut oil, coconut water, folic acid, Lugols Iodine, aloe vera water, potassium nitrate, potassium sorbate, vitamin C, CoEnzyme Q10, cranberry, bioflavonoids, beta-carotene, vitamin E, selenium, Himalayan sea salt, activated charcoal, neem (margosa), apple cider vinegar, theobromine, malic acid, blueberries, silver, egg shells, witch hazel, bamboo, vegetable glycerin, calendula officinalis flower extract, golden seal extract, ginseng root extract, perilla ocymoides seed extract, papaya extract, Melissa officinalis leaf extract, corn mint oil, benzoic acid (natural), almond oil, grapefruit seed extract, ozonized water, lemon juice, clove, cinnamon, citrus oil, rosemary extract, sage extract, lemon balm extract, green tea extract, kaempferia galangal extract, neem leaf extract, isolated phytochemicals such as cinnamates, benzoates, and eugenol, any other suitable ingredient, or any combination thereof.
Zinc: Some studies indicate that many people's diets may be deficient in zinc. Chronic diseases, medications, lifestyle, and age may also contribute to overall zinc deficiency. In various embodiments, zinc may be provided in the form of zinc oxide. In some embodiments, zinc may be provided in the form of a zinc sulfate liquid. However, it may be appreciated that any suitable source of zinc may be used. Zinc may reduce mouth odors, help control plaque thereby inhibiting tartar (calculus) formation, and assist in the remineralization of teeth and bone. In addition, zinc can play an important role in maintenance of a healthy immune system. A compromised immune system may generally open the body, and more specifically open the oral cavity, to infection. Zinc may also be required in the manufacture of the male hormone testosterone. Zinc may also be a strong aromatase inhibitor which decreases the natural biological conversion of testosterone to the female hormone estrogen in males.
Potassium: In some embodiments, potassium may be provided in the form of potassium gluconate. In some embodiments, potassium may be provided in the form of potassium citrate. In some embodiments, potassium may be provided in the form of potassium sorbate. In some embodiments, potassium may be provided in the form potassium bicarbonate, which may additionally help to regulate pH. However, it may be appreciated that any suitable source of potassium may be used. Potassium may be used to aid the retention of calcium in the bloodstream. Decreased blood levels of calcium may result in the pulling of calcium from bone (including dental bone and teeth) in order for the body to maintain the proper level of calcium in the blood. Some studies have correlated low serum potassium levels with a higher incidence of periodontal disease and bone loss. Similarly, increased urinary excretion of potassium has been tied to an increase in the severity of periodontitis, a chronic inflammatory process. Chronic inflammation is believed to lead to an increase in hypertension. Potassium can also play a significant role in the regulation of nearly all bodily functions including cardiovascular, muscular, and neurological processes. In males, there has been research and documentation that testosterone may regulate potassium levels by facilitating the opening of potassium channels. Conversely, there is theory that a deficiency of potassium may lead to less testosterone as there is less need for potassium opening channels. Additionally, potassium sorbate may be used to help control the formation of yeast, fungi, and/or mold in the oral cavity. Potassium sorbate is a potassium salt of sorbic acid, which is a naturally occurring substance that may inhibit growth of yeast, fungi, and/or mold.
Vitamin D: Vitamin D may be beneficial for many reasons. It has been estimated that a significant portion of the population may be deficient in vitamin D. This is despite the use of vitamin D as a nutritional supplement in many foods, beverages, and other products, not to mention the natural production of vitamin D through exposure to sunlight. As is the case with potassium, vitamin D deficiency can also be attributed to chronic diseases, medications, lifestyle, and age. Vitamin D may be necessary as a modulator of calcium absorption. Calcium may be a vital component in the production of strong bone tissue, including jawbone and tooth bone. Vitamin D has been linked to controlling the gene which may be directly responsible for periodontitis. Vitamin D may be an anti-inflammatory agent which in itself helps decrease the inflammation associated with gum disease. Vitamin D may be a regulator in the production of cathelcidin and defensin, both antimicrobic proteins which help fight the bacteria which produce tooth decay and periodontal disease. It may also reduce the levels of matrix metalloproteinases, which are enzymes associated with periodontal disease. Also, vitamin D in males may improve virility as a direct correlation has been shown between summer seasonal highs of vitamin D, through increased exposure to sunlight, and measured male virility. In addition, some endocrological studies have indicated a possible correlation between increased intake of vitamin D and a lower body weight. Other studies have linked the seasonal summer peak of naturally occurring 25 (OH) vitamin D levels due to increased exposure to sunlight with a peak in the physical fitness levels of male athletes. In addition, vitamin D may be a necessary modulator in the absorption of calcium, especially in women, in which osteoporosis and related diseases have been found more prevalent. There may also be links of vitamin D deficiency with heart disease, diabetes, and other disease states.
Vitamin K: Vitamin K is used by the body for bone growth, and it may help transport dietary calcium and remove excess calcium from tissues. In some embodiments, Vitamin K may be provided as Vitamin K2. Vitamin K2 may help direct calcium to bones, while preventing it from being deposited in other locations, such as in organs, joint spaces, and arteries. Vitamin K2 and Vitamin D3 may work together to support bone health, and thus formulations of the present disclosure may have both components in some embodiments.
Phosphorus: Phosphorus is the second most abundant mineral in the human body which plays a role in many of the body's chemical reactions. Phosphorus may added to help support overall health.
Magnesium: Magnesium aids in the body's absorption of calcium and also plays a key role in the strength and formation of bones and teeth. Magnesium helps prevent abnormal blood clotting and also helps maintain proper muscle function. Magnesium and phosphorus may collaborate with calcium to mineralize bones and teeth; magnesium might also work with potassium to prevent blood from becoming too acidic, which can leach calcium from bones. Some chemical reactions in the body, including bone and mineral metabolism, rely on magnesium. Magnesium may be provided to support these functions.
Calcium: Some studies have indicated that the average diet may also be deficient in calcium, as compared to the recommended daily allowance (RDA). This correlates well with studies that have shown that as many as around half of women are calcium deficient. Calcium is typically added as a dietary supplement to foods, beverages, and many vitamin and nutritional products. Further calcium deficiency can be attributed to chronic diseases, medications, lifestyle, and age. The enamel layer of tooth material has been measured to comprise a significant amount of calcium. Decreased blood levels of calcium may result in the pulling of calcium from bone (including dental bone and teeth) in order for the body to maintain the proper level of calcium in the blood. Remineralization of tooth material by direct absorption of calcium may result in stronger teeth and may also facilitate the repair of damaged teeth. Some studies have also shown a link between low calcium levels and periodontal disease. Periodontal disease includes the inflammation and degeneration of the bone and gum structure which support the teeth. As previously discussed, this chronic inflammatory may lead to an increase in hypertension. Hypertension may be a risk factor in the development of cardiovascular disease, which has been a leading cause of deaths in women. In some embodiments, calcium may be used as a mild abrasive in, for example, toothpaste formulations, to help scrub stains and unwanted particles from tooth enamel surfaces. In some embodiments, calcium may be provided in the form of calcium citrate or coral calcium.
Vitamin B compounds: One vitamin B compound is vitamin B6. In some embodiments, vitamin B6 may be provided using pyridoxine. In some embodiments, vitamin B6 may be provided using a pyridoxine HCl liquid. However other suitable chemical compounds may be used to create or supply a vitamin B compound, and/or more specifically vitamin B6. Some studies have indicated that vitamin B6 may be severely lacking in most diets, and more particularly in female diets. Also diets high in sugar intake can lead to a deficiency of vitamin B6, as it is needed at additional levels to metabolize sugar. Additionally, chronic diseases, medications, lifestyle, and age can be contributory factors to vitamin B6 deficiency. Specifically, with females, normal monthly hormonal changes associated with the female reproductive system may contribute to a deficiency in vitamin B6. Vitamin B6 deficiency can lead to sore mouth and mouth lesions as well as impaired healing of those type sores and lesions. Deficiency may also lead to a glossitis of the tongue which is commonly referred to as “Smooth Tongue.” Smooth Tongue may often manifest as inflammation with loss of lingual papillae which may result in a smooth reddened surface. Vitamin B6 may also be essential in production of hemoglobin. Hemoglobin may carry oxygen in the blood. Vitamin B6 deficiency and the resulting loss of oxygen in the blood supply to the teeth and soft tissues of the oral cavity can lead to decay and even death of teeth. In addition, deficiency of vitamin B6 may play an important role in facial oiliness, increased fatigue, hair loss, nausea, and other acute and chronic conditions. In some embodiments, vitamin B may be provided in the form of vitamin B12. Vitamin B12 has been shown to provide energy.
Magnesium: In some embodiments, magnesium may be provided in the form of magnesium oxide. In some embodiments, magnesium may be provided in the form of a magnesium chloride liquid. However, it may be appreciated that any suitable source of magnesium may be used. Some studies have shown that the average diet may also be deficient in magnesium, as compared to the RDA. Even though it may be an essential component of over 300 chemical reactions in the human body, magnesium is often unrecognized as a vital nutrient. Magnesium may stabilize calcium in the blood so that it can be properly incorporated in the structure of bone, including jaw bone and teeth. In addition to calcium, magnesium may be another mineral essential for bone health. It may also play a role in the direct remineralization of tooth material. Some research has shown increased magnesium levels may be directly associated to a decrease in attachment loss of teeth. Additional studies have directly link increased magnesium levels to a decrease in inflammation—a component of periodontal disease. Magnesium may additionally play a role in cardiovascular health and in prevention of diabetes.
Sodium Bicarbonate: Sodium bicarbonate may be a natural tooth whitener. It may also have antimicrobial properties. Sodium bicarbonate may help neutralize acids which are the byproduct of bacteria metabolism. These acids may erode the enamel surface of a tooth which leads to cavities. Additionally, sodium bicarbonate may help to neutralize acidic conditions within the oral cavity caused by other sources. Sodium bicarbonate, when mixed with water, may release free radicals which may break down stain molecules and allow them to be removed from dental surfaces during normal brushing. In some embodiments, potassium bicarbonate may be used instead of, or in addition to, sodium bicarbonate. Potassium may promote overall body health. It has been shown that many individuals are deficient in daily potassium intake.
Xylitol: In at least one embodiment, xylitol may be provided in the form of birch tree xylitol. However, any suitable source for xylitol may be used. Decay causing bacteria inside the mouth may feed on ingested sugars and secrete a strong acid as a metabolic byproduct. These acids in turn may attack the enamel dental surface and erode it, leading to cavities. Xylitol is a complex natural, non-fermentable sugar which may not be metabolized by bacteria. The bacteria may not be able to feed and as a result die, thus ending the acid production and erosion cycle. In some cases, using products with xylitol can reduce levels of streptococcus mutans and/or other bacteria by 27-75%, without harming beneficial bacteria. Xylitol may also have a very sweet taste, which leads to an increased salivary flow. Increased saliva may produce an optimal pH in the oral cavity and further neutralizes decay causing acids. Saliva contains calcium and phosphate, which may aid in remineralization of teeth. Xylitol may also reduce the acidity of saliva, which may help to fight acid-driven degradation of tooth enamel. Xylitol may increase absorption of calcium in the digestive system. Xylitol may reduce cavities and tooth decay by as much as 30-85%. Xylitol may be especially active against Strep Mutans, a bacterium known to cause cavities. In some embodiments, Xylitol may be added as an antibacterial and/or sweetening agent to products of the present disclosure.
Xanthum Gum: Xanthum gum may be made by combining fermented sugars with a specific type of bacteria. The resulting compound may be used as a stabilizing agent in foods and medicinal products. In various embodiments, xanthum gum, in combination with one or more essential oils, may be used as a suspending agent or emulsifier.
Coconut oil: A primary component of coconut oil may be Lauric acid. Lauric acid may be an antimicrobial, antiviral, and antifungal. Coconut oil may also contain antioxidants that may be effective in reducing inflammation. In some embodiments, coconut oil may serve as a carrier to facilitate the incorporation of one or more essential oils into a product, such as toothpaste. Additionally, coconut oil may attract and/or encapsulate bacteria in the mouth, such that the bacteria may be expelled by, for example, spitting and/or rinsing.
Coconut water: Coconut water may be the clear liquid extracted from the inside of young green coconuts. Coconut water may contain high levels of vitamin C, several B vitamins, and/or minerals calcium, magnesium, potassium, sodium, and/or phosphorus. Vitamin C has been shown to increase immunity and decrease inflammation. The presence of multiple minerals may help to maintain a balanced pH and neutralize the acids produced by decay producing bacteria. Coconut water may contain Lauric acid which may be an antimicrobial, antifungal, and antiviral. It may also contain high concentration of cytokinins, known to be powerful antioxidants.
Folic Acid: Some studies have shown that the average diet may also be deficient in folic acid, as compared to the RDA. Chronic diseases, medications, lifestyle, age, and/or pregnancy may also contribute to deficiency of folic acid. Some studies have additionally indicated significant deficiency rates in women, from adolescent girls up to elderly women. Folic acid may act as a modulator of cell growth and cell repair. Mouth sores and tongue swelling may be oral manifestations of folic acid deficiency. Some recent research indicates that used topically as a mouth rinse, folic acid can actually improve the body's oral defenses. Its action as a cell repair modulator has been shown to decrease the risk of gingivitis and periodontitis. Folic acid may also decrease the inflammatory response of gum tissue. It may also decrease bleeding in people with gingivitis. In addition, folic acid has been documented to protect against heart disease, birth defects, and anemia.
Lugols Iodine: In some embodiments, Lugol's iodine may be presented in the form of a Lugol's solution. For example, Lugols Iodine may be a solution with 1-5% elemental iodine. As may be appreciated, a Lugol's solution may contain less than 1% or greater than 5% elemental iodine. In various embodiments, the Lugol's solution may also contain potassium iodide in water. For example, in at least one embodiment containing 5% elemental iodine, the solution may also contain 10% potassium iodide in water. As may be appreciated, the various Lugol's solutions may contain 10%, less than 10%, or greater than 10% potassium iodide. In some embodiments, the elemental iodine and potassium iodine may be mixed with distilled or purified water. Lugols Iodine may be used as an antimicrobial and antifungal. Other uses for Lugols Iodine may include emergency water disinfection and thyroid gland protection against radiation exposure.
Aloe Vera Water: Aloe vera water may be the cold pressed liquid extracted from the aloe vera plant. Some studies have shown that aloe vera water may possess antimicrobial, antifungal, and virucidal properties. Some research has also noted that aloe vera water may possibly be effective in the treatment of gingivitis and some inflammatory mouth conditions.
Potassium nitrate: Potassium nitrate may be used to decrease hypersensitivity. In particular, potassium nitrate may block synapses between nerve cells, thereby decreasing nerve excitation and pain. Thus, potassium nitrate may be used in formulas for sensitive teeth, such as sensitive toothpaste.
Vitamin C: Vitamin C has been shown to be an anti-oxidant. Vitamin C has also been shown to be helpful in connective tissue repairs. Vitamin C has even been shown to accelerate bone regeneration.
Bioflavonoids: Bioflavonoids may contain Vitamin C. In addition, bioflavonoids may be important for collagen structure, which helps to reduce gum inflammation.
CoEnzyme Q10, or CoQ10: CoEnzyme Q10 may be a natural anti-oxidant. CoEnzyme Q10 may be helpful to the proper repair of gum tissue.
Cranberry, or some derivative thereof: Cranberry may prevent bacteria from sticking to teeth.
Beta-Carotene, or vitamin A: Beta-carotene may be helpful in maintaining periodontal tissue and improving immune function. In some embodiments, beta-carotene may be used to help in collagen synthesis. In some embodiments, beta-carotene may be used to aid in wound healing.
Vitamin E: Vitamin E has been shown to be an anti-oxidant. In various embodiments, vitamin E may be used to help promote wound healing. In some embodiments, selenium may, additionally or alternatively be used in various product formulas. Selenium has been shown to work in synergy with vitamin E as an anti-oxidant.
Himalayan Sea Salt: In various embodiments, Himalayan sea salt may be used as a tooth whitener. In some embodiments, Himalayan sea salt may, additionally or alternatively, be used for remineralization.
Activated Charcoal: In at least one embodiment, charcoal or activated charcoal may be used to add a natural color to one or more products, discussed herein. In some embodiments, charcoal or activated charcoal may additionally or alternatively be used as a whitener.
Apple Cider Vinegar: Apple cider vinegar has been shown to act as an antimicrobial. In some embodiments, apple cider vinegar may, additionally or alternatively, be used as a tooth whitener.
Theobromine: Theobromine may, in some embodiments, be used as a tooth whitener. In some embodiments, theobromine may, additionally or alternatively be used to help harden teeth.
Malic acid: In at least one embodiment, malic acid may be extracted from strawberries, apples, or other fruits. Malic acid may, in some embodiment, be used as a tooth whitener. Additionally, malic acid may have a natural tartness that may help to stimulate the production of saliva to help wash away harmful bacteria in the oral cavity.
Blueberries: Blueberries, blueberry juice, or some other blueberry derivative may be used to add a natural color to one or more products, discussed herein. In some embodiments, blueberries, or some derivative thereof, may additionally or alternatively be used to whiten teeth.
Silver: Silver may be a natural antibacterial and/or antiseptic. In various embodiments, silver may be provided in the form of ionic silver. In at least one embodiment, silver may, additionally or alternatively, be provided in the form of colloidal silver. Silver may help to inhibit biofilm.
Benzoic Acid: In some embodiments, benzoic acid may act as an antifungal and antiseptic.
Ground up Egg Shells: In at least one embodiment, organic egg shells may be used. Egg shells may be used as an antibacterial and antiviral. In some embodiments, various minerals found in egg shells may also be used to replenish one or more mineral deficiencies. In various embodiments, ground up egg shells may, additionally or alternatively, act as an abrasive.
Witch Hazel: Witch Hazel, or Hamamelis water, may be alcohol-free. Witch hazel may have antioxidant and/or antitumor properties. In some embodiments, witch hazel may, additionally or alternatively, be used as an astringent. Witch hazel may also have anti-inflammatory properties. Witch hazel may also be used as an emulsifier.
Bamboo: In some embodiments, bamboo may be provided in the form of bamboo fiber. In other embodiments, bamboo may be provided in the form of a bamboo extract. As may be appreciated, any suitable form or derivative of bamboo may be used in the various embodiments disclosed herein. Bamboo may have beneficial stain removing properties
Vegetable Glycerin: Vegetable glycerin may be used as an emollient in some embodiments. The vegetable glycerin may generally prevent or mitigate a product or composition from drying out.
Diatomaceous Earth: Diatomaceous earth is a naturally occurring mineral rock. It is formed from the fossilized remains of diatoms. Diatomaceous earth may include silica. Silica may be used, in some embodiments of the present disclosure, as a mild abrasive to help scrub stains and unwanted particles from tooth enamel surfaces.
Xanthan Gum: Xanthan gum is the end product of a fermentation process of simple sugars and Xanthomonas campestris species of bacteria. Xanthan gum may be used as an emulsifier to prevent separation of components.
Arrowroot Flour: Arrowroot flour is a starch derived from the roots of the

Arrowroot plant. Arrowroot flour may be used as an enhancer to provide improved and/or more uniform consistency.
Water: Formulations of the present disclosure may include water, which may be distilled water or purified water, for example. For example, water may be purified through filtration, osmosis, and/or UV light exposure processes.
Phosphate: Phosphate may help to promote reformation of hydroxyapatite within the oral cavity.
Additional Vitamins and/or Minerals: Some vitamins and minerals are needed in the human body to help some metabolic and physiologic processes within the human body. Incorporating the above-described vitamins and/or minerals, and/or others, into formulations of the present disclosure may help lower the susceptibility of degradation or demineralization of tooth enamel.

Any of the oral care product formulations of the present disclosure may use or incorporate, or otherwise comprise, any one or more of the above-identified essential oils or other ingredients, alone or in combination. For example only, several essential oils may be used to strengthen or tighten gums. In various embodiments, the essential oils that may strengthen gums may include, but are not limited to, benzoin, cypress, lemon, lemongrass, and peppermint.

Some essential oils and other antimicrobials may act as preservatives in some formulations of the present disclosure. For example, some plants, herbs, and/or essential oils thereof that may provide antimicrobial as well as preservation properties include, but are not limited to, basil, caraway, cardamom, cinnamon, clove, coriander, cumin, eucalyptus, fennel, garlic, grape, green tea, lavender, lemon, marjoram, mint, mustard, nutmeg, onion, oregano, peppers, rose, rosemary, sage, sandalwood, and thyme. Grapefruit seed extract and citricidals are antimicrobials that may also be used as preservatives in some embodiments. Moreover, some antimicrobials from microorganisms that may be used as preservatives include, but are not limited to, acidopholin, bulgaricin, colicins, helveticin, lactacin, natmycin, nisin, pediocin, plantaricin, and reuterin. Some antimicrobials from animals that may be used as preservatives include, but are not limited to, chitosan, defensins, lactoferrin, lactoperoxidase system, lysozyme, and pleurocidin. Paraben-based preservatives may be used in some products of the present disclosure. Paraben based preservatives include, but are not limited to, germaben, germaben II, phenonip, methyparaben, germall, and liquapar oil.

Toothpaste

In accordance with the present disclosure, one or more unique toothpastes may be created. In various embodiments, the toothpaste may be comprised of one or more ingredients listed above or elsewhere herein. Toothpaste formulations of the present disclosure may also contain baking soda, which has been shown to be extremely effective in cleaning and polishing teeth. In various embodiments, the baking soda may be naturally derived from the earth, rather than chemically processed. In addition, the toothpaste may contain aloe vera and/or sea salts.

In one embodiment of the present disclosure, a toothpaste may be formulated with zinc oxide, potassium gluconate, vitamin D, magnesium oxide, sodium bicarbonate, xanthum gum, coconut oil, distilled water, baking soda, and/or any other suitable ingredients. In an additional or alternative embodiment, a toothpaste may contain one or more or all ingredients including, but not limited to, distilled water, coconut water, aloe vera juice, birch tree xylitol, zinc citrate, potassium, vitamin D, magnesium chloride, baking soda, Himalayan sea salt, and/or one or more essential oils. In at least one embodiment, toothpaste may have a flavor profile that is minty, or intensely mint. In order to obtain a minty or intense mint flavor, one or more essential oils may be used including, but not limited to, wintergreen, spearmint, peppermint, or mint. In various embodiments, other flavors may be created, for example and example only, using cinnamon, clove, and/or tea tree essential oils.

In another embodiment, a toothpaste may be formulated with calcium citrate, vitamin D, vitamin B6, magnesium oxide, xylitol, xanthum gum, coconut oil, distilled water, baking soda, and/or any other suitable ingredient(s). In various embodiments, one or more essential oils may be used. For example, a toothpaste may be formulated to include mint, spearmint, vanilla, and/or tea tree essential oils.

In another embodiment, a toothpaste may be formulated with birch tree xylitol, calcium citrate, diatomaceous earth, malic acid, coconut oil, potassium sorbate, xanthan gum, such as organic xanthan gum, arrowroot flour, such as organic arrowroot flour, water such as purified water, and/or other suitable ingredient(s). In various embodiments, one or more essential oils may be used. For example, a toothpaste may be formulated to include lemon, lime, sweet orange, wintergreen, spearmint, and/or peppermint essential oils.

In at least one embodiment, toothpaste may be designed for sensitive teeth. In one such embodiment, the toothpaste may additionally or alternative include, but is not limited to, potassium nitrate and/or essential oils such as clove oil which may serve as an analgesic.

In yet another embodiment, a toothpaste may be designed to facilitate whitening of teeth. In one such embodiment, a toothpaste may include, but is not limited to, malic acid, baking soda, and/or lemon essential oil. In various embodiments, natural ingredients such as charcoal or blueberries may, additionally or alternatively, be used to whiten and/or to add a natural color(s) to one or more toothpaste formulas contemplated herein. As may be appreciated, one or more other ingredients used to whiten teeth and/or color the toothpaste may, additionally or alternatively, be used.

Various toothpaste formulations of the present disclosure may generally reduce and/or substantially eliminate any residue left on teeth after brushing as compared to conventional toothpastes. This can be due, in part, to a reduction or elimination of glycerin in the embodiments of the present disclosure.

In one embodiment, a toothpaste of the present disclosure may include xylitol, calcium, such as calcium citrate or coral calcium derived from deposits of coral reefs, diatomaceous earth, malic acid, xanthan gum, arrowroot flour, coconut oil, one or more essential oils, potassium sorbate, and water such as distilled or purified water. For example, the toothpaste may have between about 1% and about 70% by volume of xylitol, such as Birch tree xylitol, in some embodiments. In particular, the toothpaste may have between about 10% and about 50%, or between about 20% and about 35%. The toothpaste may have between less than 1% and about 30% by volume of calcium, such as calcium citrate or coral calcium, in some embodiments. Particularly, the toothpaste may have between about 1.5% and about 20%, or between about 3% and about 15%, by volume of calcium citrate or coral calcium. The toothpaste may have between less than 1% and about 24% by volume of diatomaceous earth in some embodiments. In particular, the toothpaste may have between about 1.5% and about 18%, or between about 2% and about 12%, by volume of diatomaceous earth. The toothpaste may have between about 0.1% and about 10% by volume of malic acid in some embodiments. In particular, the toothpaste may have between about 0.5% and about 7.5%, or between about 1% and about 5%, by volume of malic acid. The toothpaste may have between less than 1% and about 30% by volume of xanthan gum in some embodiments. Particularly, the toothpaste may have between about 1.5% and about 20%, or between about 2% and about 15%, by volume of xanthan gum. The toothpaste may have between less than 1% and about 28% by volume of each of coconut oil and arrowroot flour. In particular, the toothpaste may have between about 1.5% and about 21%, or between about 2% and about 14%, by volume of coconut oil and/or arrowroot flour. The toothpaste may have between less than 0.001% and about 8% by volume of one or more essential oils. Particularly, the toothpaste may have between about 0.005% and about 6%, or between about 0.01% and about 4%, by volume of one or more essential oils. The one or more essential oils may comprise a blend of oils. For example, the one or more essential oils may include a blend of any of lemon oil, lime oil, sweet orange oil, wintergreen oil, spearmint oil, peppermint oil, ylang ylang oil, vanilla oil, cinnamon oil, clove oil, grapefruit oil, eucalyptus oil, and myrrh oil. Other oils and/or blends of oils may be used in other embodiments. The toothpaste may have between less than 0.05% and about 5% by volume of potassium sorbate in some embodiments. In particular, the toothpaste may have between about 0.075% and about 3.75%, or between about 0.1% and about 2.5%, by volume of potassium sorbate. The toothpaste may have between less than 0.01% and about 4% by volume of each of potassium, magnesium, and phosphorus, individually. In particular, the toothpaste may have between about 0.025% and about 3%, or between about 0.05% and about 2% of each of potassium, magnesium, and phosphorus, individually. In some embodiments, the toothpaste may have between less than about 0.001% and about 1% by volume of each of vitamin K and vitamin D, individually. Particularly, the toothpaste may have between about 0.005% and about 0.75%, or between about 0.01% and about 0.5% of each of vitamin K and vitamin D, individually. The toothpaste may have between less than about 0.001% and about 1% by volume of one or more trace minerals, or between about 0.005% and about 0.75%, or between about 0.01% and about 0.5%. In some embodiments, the toothpaste may have between less than 0.001% and about 8% by volume of one or more vitamins and between less than 0.001% and about 8% by volume of one or more minerals. In particular, the toothpaste may have between about 0.005% and about 6%, or between about 0.01% and about 4% of one or more vitamins, and the toothpaste may have between about 0.005% and about 6%, or between about 0.01% and about 4% of one or more minerals. The toothpaste formulation, according to one or more embodiments, is shown in the table below.

| Ingredient | Quantity (% by volume) |
|---|---|
| Birch Tree Xylitol | 20-35% |
| Calcium Citrate | 3-15% |
| Diatomaceous Earth | 2-12% |
| Malic Acid | 1-5% |
| Organic Xanthan Gum | 2-15% |
| Organic Arrowroot Flour | 2-14% |
| Coconut Oil | 2-14% |
| Essential Oil Blend (Lemon Oil, Lime Oil, | 0.01-4% |
| Sweet Orange Oil, Ginger Oil, Tea Tree | |
| Oil, Wintergreen Oil, Spearmint Oil, | |
| Peppermint Oil, Ylang Ylang Oil, Vanilla | |
| Oil, Cinnamon Oil, Clove Oil, Grapefruit | |
| Oil, Eucalyptus Oil, Myrrh Oil) | |
| Potassium Sorbate | 0.1-2.5% |
| Potassium | 0.05-2% |
| Magnesium | 0.05-2% |
| Phosphorus | 0.05-2% |
| Vitamin K | 0.01-0.5% |
| Vitamin D | 0.01-0.5% |
| Trace Minerals | 0.01-0.5% |
| Other Vitamins | 0.01-4% |
| Other Minerals | 0.01-4% |
| Purified Water | Balance |

In some embodiments, additional and/or alternative ingredients may be used in a toothpaste of the present disclosure. Moreover, a toothpaste of the present disclosure is not limited to the quantities described above.

Of course, any combination of ingredients may be used and/or tailored for particular needs or goals. Furthermore, any combination of ingredients may be used and/or tailored for any event, occasion, or experience as discussed more below. As may be appreciated, other combinations of ingredients may yield other suitable toothpastes. As may also be appreciated, other ingredients may be used to add to the taste, smell, color, etc. of the toothpaste.

Mouthwash or Rinse

In accordance with the present disclosure, one or more unique mouthwashes (herein referred to interchangeably as rinses) may be formulated or created. In various embodiments, the mouthwashes may be comprised of one or more ingredients listed above or elsewhere herein to support the distinct oral ecology of a user's mouth. In various embodiments, the mouthwash formulas may be alcohol-free. For example, in one such embodiment, witch hazel may be used as an emulsifier. In some embodiments, the mouthwash formulas may contain essential oils and/or natural botanicals designed to help control tartar buildup. In various embodiments, the mouthwash formulas may also contain baking soda, which may restore an optimal pH of the mouth, thereby protecting tooth enamel, and making the mouth less hospitable to bacteria growth.

In one embodiment of the present disclosure, a mouthwash may be formed using zinc sulfate liquid, potassium citrate, magnesium chloride liquid, Lugol's iodine, xylitol, coconut water, aloe vera water, distilled water, and/or one or more essential oils. In various embodiments, a non-limiting list of essential oils used in mouthwash may include peppermint, orange, spearmint, and/or lemon. In another such embodiment of the present disclosure, another mouthwash may be formed using vitamin B6, folic acid, magnesium, Lugol's iodine, xylitol, coconut water, aloe vera water, distilled water, and one or more essential oil extracts. In one embodiment of the present disclosure, a mouthwash formulated to whiten teeth may be formed using malic acid, baking soda, lemon oil, and/or any other suitable ingredients. In various embodiments, a non-limiting list of essential oils used in mouthwash may include, mint, lemon, peppermint, orange, and/or spearmint. As may be appreciated, these formulations are non-limiting examples of products contemplated by the present disclosure.

The various mouthwashes or rinses may also be formulated to include any one or more of the ingredients identified herein or combinations thereof, and for achieving similar purposes as described herein with respect to other oral care products. The various mouthwashes may be formulated for a consumer's unique preferences or needs. Some non-limiting examples may include, but are not limited to, a mouthwash or rinse tailored to prevent or fight gum disease, fight stains and/or whiten, and/or for use with sensitive teeth. In one embodiment designed to fight or prevent gum disease, potential ingredients may include, but are not limited to, vitamin C, CoQ10, bioflavonoids, vitamin E, selenium, beta-carotene, and/or tea tree oil. In another embodiment designed to fight stains and whiten, potential ingredients include, but are not limited to, neem, apple cider vinegar, activated charcoal, Himalayan sea salt, theobromine, and/or zinc oxide. In still another embodiment designed for users with sensitive teeth, a rinse may potentially include, but is not limited to, potassium nitrate.

Rinses may also be formulated to be used at different times during the day. For example, one rinse may be formulated for use during the morning while another rinse may be formulated for use at night. In one embodiment, potential ingredients for an AM rinse may include, but are not limited to, vitamin B12, chromium, and/or vitamin C. In such an embodiment, the vitamin B12 may provide energy, the chromium may boost metabolism, and the vitamin C may act as an anti-oxidant. In contrast, another embodiment designed for use at night may contain potential ingredients including, but not limited to, lavender oil and/or chamomile. Lavender and chamomile may have a soothing or calming effect. As may be appreciated, other combinations of ingredients may yield other suitable mouthwashes.

In one embodiment, a mouth wash or rinse of the present disclosure may include xylitol, one or more essential oils, potassium sorbate, and water such as purified or distilled water. For example, the mouth wash or rinse may have between less than 1% and about 60% by volume of xylitol, such as Birch tree xylitol, in some embodiments. In particular, the mouth wash or rinse may have between about 4% and about 45%, or between about 7% and about 30%, by volume of xylitol. The mouth wash or rinse may have between less than 0.1% and about 7% by volume of one or more essential oils in some embodiments. In particular, the mouth wash or rinse may have between about 0.15% and about 5%, or between about 0.2% and about 3.5%, by volume of one or more essential oils. The one or more essential oils may comprise a blend of oils. For example, the one or more essential oils may include a blend of any of lemon oil, lime oil, sweet orange oil, wintergreen oil, spearmint oil, peppermint oil, ylang ylang oil, vanilla oil, cinnamon oil, clove oil, grapefruit oil, eucalyptus oil, and myrrh oil. Other oils and/or blends or oils may be used in other embodiments. The mouth wash or rinse may have between less than 0.05% and about 1.25% by volume of potassium sorbate. In particular, the mouth wash or rinse may have between about 0.075% and about 2%, or between about 0.1% and about 2.5%, by volume of potassium sorbate. The mouth wash may have between less than about 0.001% and about 4% by volume of each of potassium, magnesium, and phosphorous, individually. In particular, the mouth wash may have between about 0.005% and about 3%, or between about 0.01% and about 2% by volume of each of potassium, magnesium, and phosphorous, individually. The mouth wash may have between less than about 0.001% and about 1% by volume of each of vitamin K and vitamin D, individually. In particular, the mouth wash may have between about 0.005% and about 0.75%, or between about 0.01% and about 0.5% by volume of each of vitamin K and vitamin D, individually. The mouth wash may have between less than about 0.001% and about 1% by volume of one or more trace minerals. In particular, the mouth wash may have between about 0.005% and about 0.75%, or between about 0.01% and about 0.5% by volume of trace minerals. In some embodiments, the mouth wash may have between less than 0.001% and about 8% by volume of one or more vitamins and between less than 0.001% and about 8% by volume of one or more minerals. In particular, the mouth wash may have between about 0.005% and about 6%, or between about 0.01% and about 4% of one or more vitamins, and the mouth wash may have between about 0.005% and about 6%, or between about 0.01% and about 4% of one or more minerals. The mouth wash or rinse formulation, according to one or more embodiments, is shown in the table below.

| Ingredient | Quantity (% by volume) |
|---|---|
| Birch Tree Xylitol | 7-30% |
| Essential Oil Blend (Lemon Oil, Lime Oil, | 0.2-3.5% |
| Sweet Orange Oil, Wintergreen Oil, | |
| Spearmint Oil, Peppermint Oil, Ylang | |
| Ylang Oil, Vanilla Oil, Cinnamon Oil, | |
| Clove Oil, Grapefruit Oil, Eucalyptus Oil, | |
| Myrrh Oil) | |
| Potassium Sorbate | 0.1-2.5% |
| Potassium | 0.01-2% |
| Magnesium | 0.01-2% |
| Phosphorous | 0.01-2% |
| Vitamin K | 0.01-0.5% |
| Vitamin D | 0.01-0.5% |
| Trace Minerals | 0.01-0.5% |
| Other Vitamins | 0.01-4% |
| Other Minerals | 0.01-4% |
| Purified Water | Balance |

In some embodiments, additional and/or alternative ingredients may be used in a mouth wash or rinse of the present disclosure. Moreover, a mouth wash or rinse of the present disclosure is not limited to the quantities described above.

Whitening Products

In accordance with the present disclosure, one or more oral care products may be formulated, using any one or more of the ingredients identified above or elsewhere herein, to whiten teeth. For example, in addition to those products mentioned previously, some non-limiting examples may include a whitening gel or paste, a whitening brush, and a whitening wand. In some embodiments, a whitening gel or paste may be used separately from, or in addition to, toothpaste in order to achieve desired results. That is, the whitening gel or paste may be applied to the brush with the toothpaste or may be applied to a brush and used at some other time. In other embodiments, a whitening gel or paste may be used in combination with a tooth whitening mouth tray. In at least one embodiment, the whitening gel or paste may be applied to disposable strips, which may be applied by the user either at home or on the go.

In accordance with the present disclosure, a tooth whitening wand may be formulated according to various embodiments. According to some embodiments, a whitening wand of the present disclosure may assist with lightening or removal of tooth stains and/or dental plaque using a wood or wood-based surface or a nano-technology sponge surface. In this regard, the whitening wand may avoid the use of whitening agents thought to be harmful or toxic. The whitening wand may be used by being rubbed against a user's teeth in an up-and-down, side-to-side, circular, or other motion. The whitening wand may be compatible with natural teeth, dentures, caps, veneers, or other types of teeth. In some embodiments, a whitening surface of the whitening wand may be infused or diffused with one or more essential oils or other materials. Essential oils may provide various benefits such as but not limited to whitening, antimicrobial, antifungal, or antibacterial effects, for example.

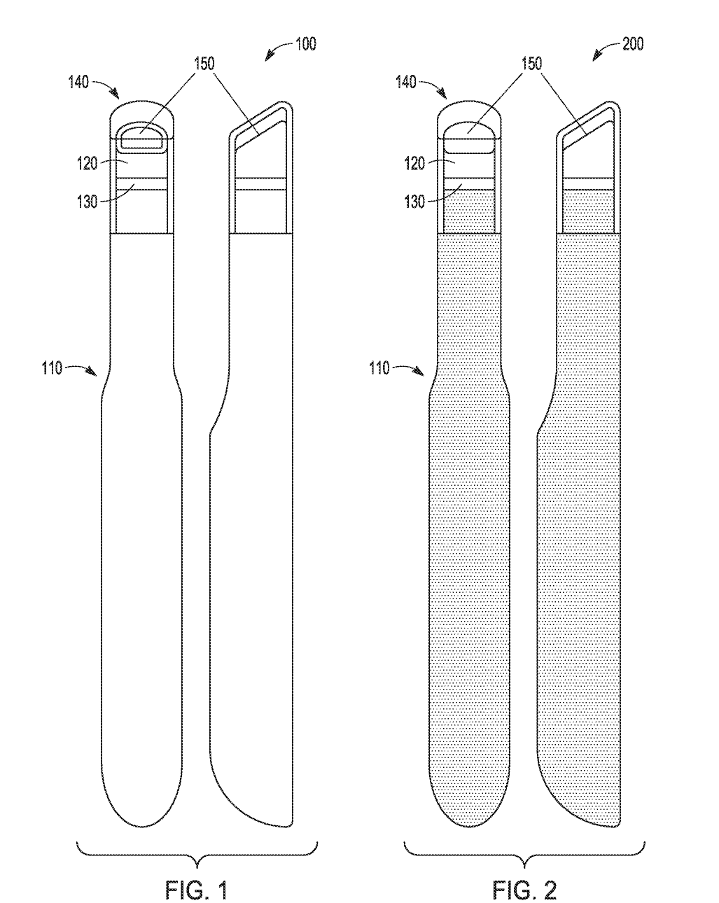

FIG. 1 illustrates a profile and front view of a whitening wand 100 of the present disclosure. FIG. 2 provides a profile and front view of a different whitening wand 200 of the present disclosure. As shown in FIGS. 1 and 2, a whitening wand generally may have a handle 110, a whitening tip 120, a tip support 130, and a cap 140.

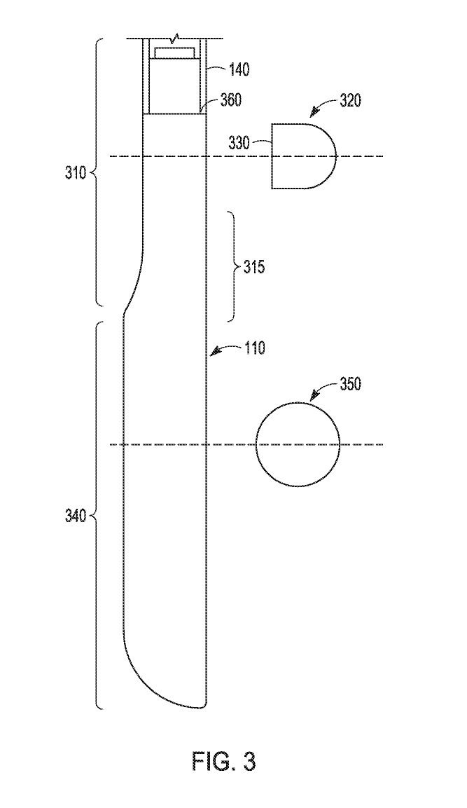

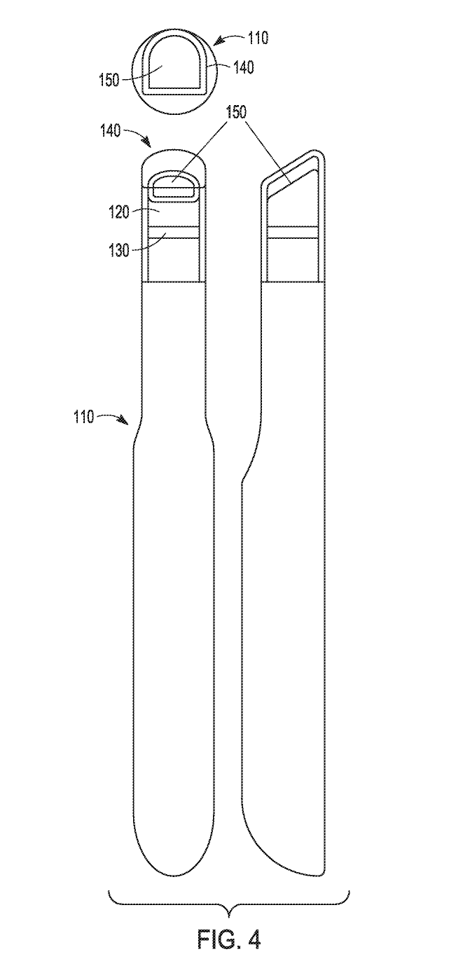

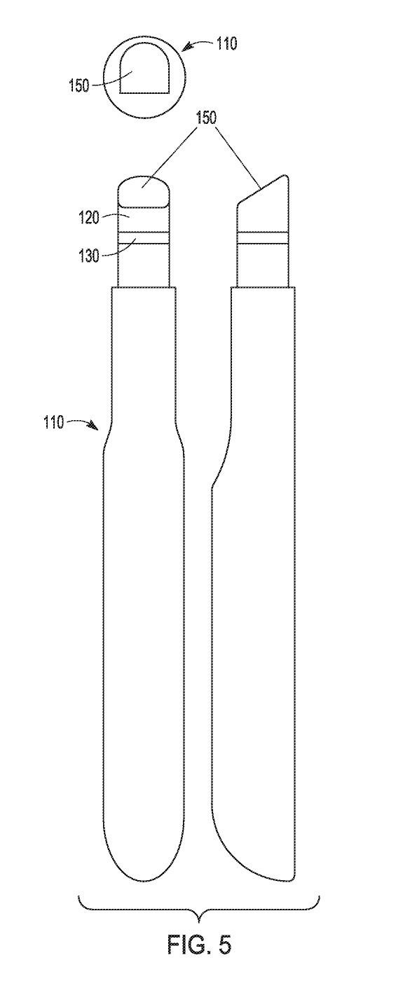

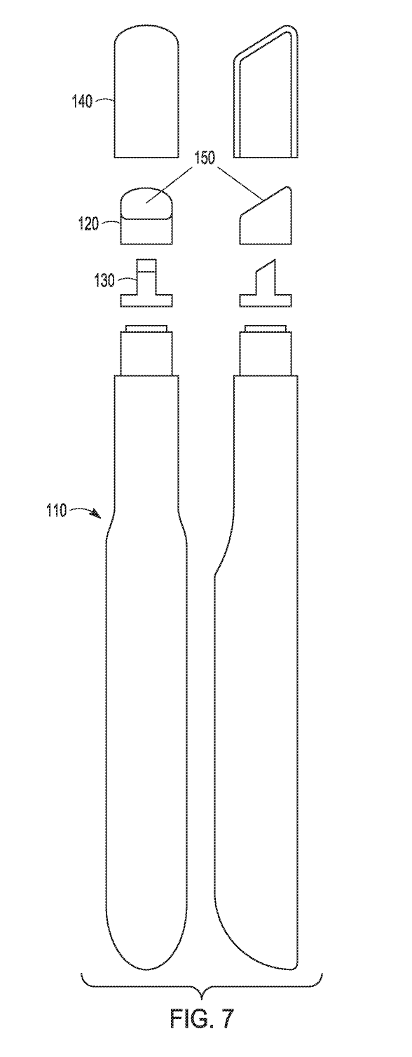

In some embodiments, the handle 110 may have at least two cross sections. As shown in FIG. 3, a first portion 310 of the handle 110 may have a first cross sectional shape 320, which may be a semi-circle, semi-oval, or other shape with a flat side 330. A second portion 340 of the handle 110 may have a second cross sectional shape 350, which may be generally circular or other suitable shape. The diameter or width of the second cross sectional shape 350, making up a majority of the handle 110, may be small enough such that a user could grasp the handle and easily maneuver the wand 100. Each cross sectional shape 320, 350 may have any diameter or width. In some embodiments, the two cross sectional shapes 320, 350 may each have a diameter or width that is between 0.25 inches and 1.5 inches. The handle 110, including both portions 310, 340, may be at least a sufficient length for a user to grasp the handle using all fingers. In some embodiments, the handle 110 may have a length that is between 3 and 10 inches long. In other embodiments, the handle 110 may have any length. The first portion 310 may contain a lip or ledge 360, which abuts the cap 140 when the cap is attached. That is, a portion of the first section 310 may fit within the cap 140. The ledge 360 may be formed by a change in the handle's 110 cross sectional area and/or shape. For example, the cross section 320 of the first portion 310 may maintain the same shape but, at a point along the first portion 310, suddenly reduce in width or diameter, the smaller width or diameter section extending distally from the second portion of the handle. This abrupt change in width or diameter may create such a ledge 360 that surrounds the perimeter of the first portion 310. The cap 140 may fit over and around this reduced cross section, such that the cap abuts with the ledge 360. In some embodiments, the diameter, width, or cross sectional area of the first portion 310 may be smaller or narrower than that of the second portion 340. Further, as discussed, the first portion 310 may have more than one diameter or width such that part of the first portion may fit within the cap 140, as shown in FIG. 3.

Where the first portion 310 meets the second portion 320 of the handle 110, as shown in FIG. 3, the cross section of the first portion may gradually increase and/or the cross section of the second portion may gradually decrease. The gradual increase of the width or diameter of the first portion 310 and/or gradual decrease of the width or diameter of the second portion 320 may form a sloped or angular transition area 315 on the handle 110 where the two portions meet, as shown in FIG. 3. This transition area 315 may allow for a user to place a thumb or forefinger on the area while holding the handle 110 during use. In some embodiments, the transition area 315 may have a raised surface or a rubber or other material so as to provide a grip, for a user's thumb or forefinger, for example. The handle 110 may be constructed of materials such as, but is not limited to, plastic, rigid plastic, metal, glass, wood, composite, fibrous materials, or some combination thereof In some embodiments, a recyclable plastic such as high or low density polyethylene or other recyclable material(s) may be used. Some portions of the handle 110 may have different densities than other portions, and some portions may be hollow. The handle 110 may be weighted at one end, such as a bottom end, located opposite from the whitening tip 120. This may allow better handling for a user. The handle 110 may have certain ergonomic features, shaping, or contouring, so as to fit comfortably within a user's hand. In some embodiments, the handle 110 may be contoured for a smaller or larger hand, such as for a female user or a male user. FIGS. 4-7 illustrate additional views and embodiments of whitening wands of the present disclosure.

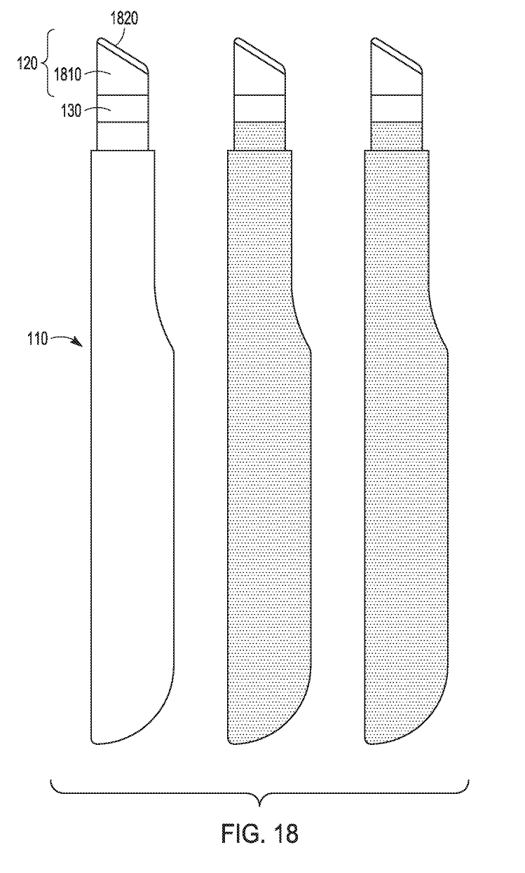

In some embodiments, the whitening tip 120 may be or include a sponge or sponge-like material. The sponge or sponge-like material may have any number of base materials for its structure. In one embodiment, the sponge may be a melamine-formaldehyde resin foam, melamine foam, or similar product. In some embodiments, the whitening tip 120 may incorporate a resin foam as described in U.S. Patent Application No. 10/882,502, filed Jul. 1, 2004, and incorporated herein by reference in its entirety. The whitening tip 120 may be constructed entirely or partially of a microencapsulated nano sponge material, according to some embodiments. A micro-compressed, nano-technology sponge may form into thousands of tiny capillaries when moistened, creating a unique 3D structure characterized by a strong, or even exceptionally strong, absorption. In other embodiments, the whitening tip 120 may be constructed of other absorbent materials and may have various capillary structures. In some embodiments, the whitening tip 120 may be composed of multiple materials or layers of materials. For example, a sponge-like material such as a microencapsulated nano sponge material may be overlaid with a layer of a different material such as a different sponge-like substance, a cotton-based material, a more rigid plastic material, or other material. The whitening tip 120 may be at least partially rigid in some embodiments, but may have any rigidity according to other embodiments. In some embodiments, as shown in FIG. 18, the whitening tip 120 may have a base 1810 that is constructed entirely or partially of a rigid plastic or other rigid material, which may be clear, partially clear, or opaque. The base 1810 may be overlaid with a layer of microencapsulated nano sponge material, melamine foam, or other sponge-like material 1820 that overlays one or more sides or surfaces of the base. In such embodiments, the base 1810 may provide additional support to the sponge or sponge-like material 1820. The layer of sponge-like or foam material 1820 may have a thickness between 0.1 and 0.5 inches in some embodiments. In other embodiments, the layer 1820 may have any thickness. The whitening tip 120 may generally have any density and thickness. The whitening tip 120 may be at least partially hollow, according to some embodiments, or may be solid or substantially solid.

In at least one embodiment, the whitening tip 120 may be constructed of a wood or wood-based material. The wood or wood-based material may generally be soft enough, and otherwise suitable for, scraping stains and/or plaque from a user's teeth without harming tooth enamel. In some embodiments, the whitening tip 120 may be constructed with bamboo, oak, willow, and/or any other suitable wood or wood-based material(s).

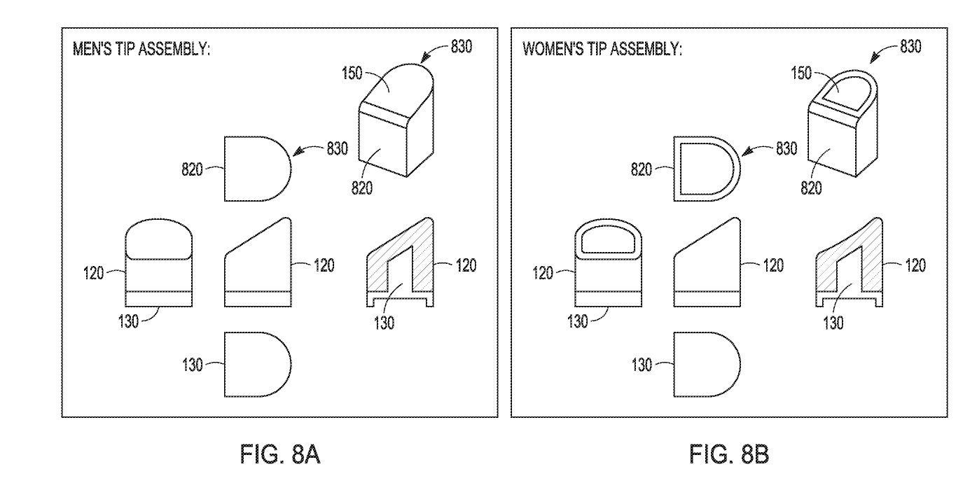

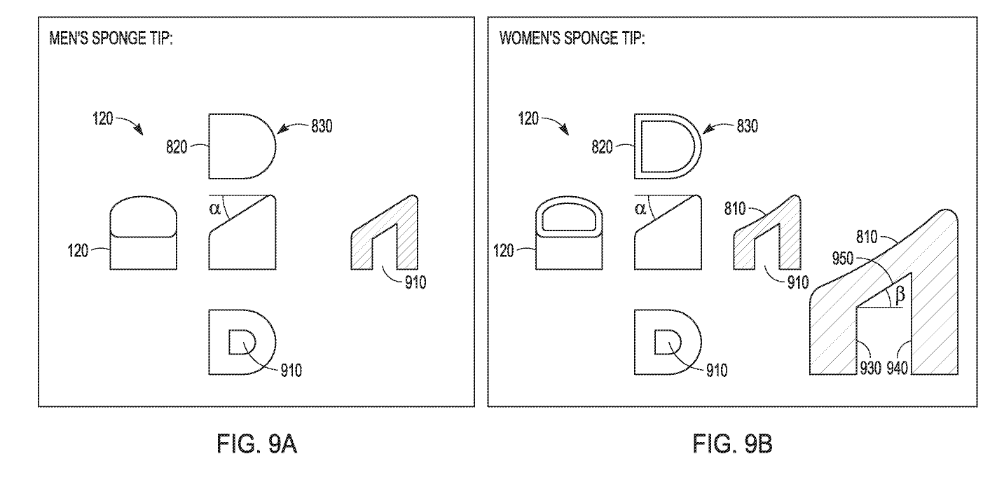

The whitening tip 120 may generally have any shape. In some embodiments, the whitening tip 120 may have a semi-circular or semi-oval cross-sectional shape, as shown in FIGS. 8, 9, and 10, or any other shape having at least one generally flat side 820. In other embodiments, the whitening tip 120 may have any suitable cross-sectional shape. The width or diameter of the whitening tip 120 may be between 0.1 and 1 inch, according to some embodiments, or may be any suitable width or diameter according to other embodiments. The whitening tip 120 may extend distally from the handle 110 or tip support 130 to generally any height. The whitening tip 120 may extend to a height of between 0.1 and 1 inch, according to some embodiments. In some embodiments, one side of the whitening tip 120, when viewed from a profile perspective, may extend further distally from the handle 110 or tip support 130 than the opposing side, as shown in FIGS. 8 and 9. A surface 150 of the whitening tip may extend at an angle a from the shorter side, upward to meet the longer side, as shown in FIG. 9. The angle a may range from 0° to 90°, but more typically from 20° to 45°, from the horizontal. The surface 150 may be designed for contact with the user's teeth. Depending on the angle a of the surface 150, a user may hold the whitening wand 100 at an angle ranging from 0° to 90°, but more typically from 20° to 45°, from a front surface of his or her teeth while using the surface 150 to clean his or her teeth. The angled surface 150 may generally be small enough to fit within a user's mouth so as to remove stains from a user's teeth. In some embodiments, the angled surface 150 may be slightly concave, as shown in FIGS. 8B and 9B. For example, where the whitening tip 120 is designed for users with small teeth or with a certain type of teeth (e.g. dentures, veneers, or others), a concave surface 810 may better form around the teeth in order to remove stains. The concave surface 810 may also allow for better detailing around the gum line or tooth edges. In some embodiments, the whitening tip 120 and/or surface 150 may be designed to fit the teeth of a female user or male user.

The whitening tip 120 may be connected to the handle 110 by way of a tip support 130. In some embodiments, the whitening tip 120 may have a bore 910 or cutout where the whitening tip connects with the tip support 130, as shown in FIG. 9. The bore 910 may have a circular cross section or may match the cross sectional shape of the whitening tip 120, and thus may have semi-circular or semi-oval shape having one straight side 920, as shown in FIG. 9. In other embodiments, the bore 910 may have any cross sectional shape. The bore 910 may have any width or diameter, but may generally have a width or diameter smaller than that of the whitening tip 120, such that the bore may be surrounded on all sides by the whitening tip. When viewed at a profile angle, the bore 910 may have a short side 930 and an opposing long side 940, wherein the long side extends further into the whitening tip 120 having a longer bore length. The short side 930 may be a straight side and the long side 940 may be rounded, according to some embodiments. At the full depth of the bore, a bottom end 950 of the bore 910 may extend at an angle β from the short side 930, upward to meet the longer side 940. The angle β may range from 0° to 90°, but more typically from 20° to 45°, from the horizontal. The bore 910 may generally be configured to accept the tip support 130.

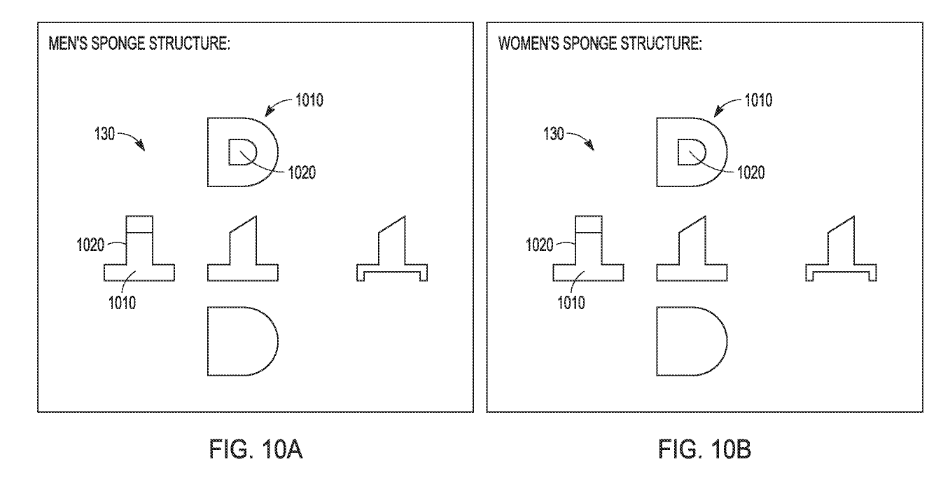

As shown in FIG. 10, the tip support 130 may consist of a base 1010 adjacent to the whitening tip 120, and a post 1020. In some embodiments, the whitening tip 120 may surround the post 1020. The post 1020 may provide the whitening tip 120 with added structural support in some embodiments. Other embodiments may include the base 1010 without a post 1020 structure. In some embodiments, one end of the post 1020 may be angled, similar to the angled surface 150 of the whitening tip 120, so as to provide support for the angled surface 150 during use. The tip support 130 may be constructed of rigid plastic or other suitable materials. In some embodiments, a recyclable plastic such as high or low density polyethylene or other recyclable materials may be used. The whitening tip 120 may be permanently, semi-permanently, or removably connected to the tip support 130.

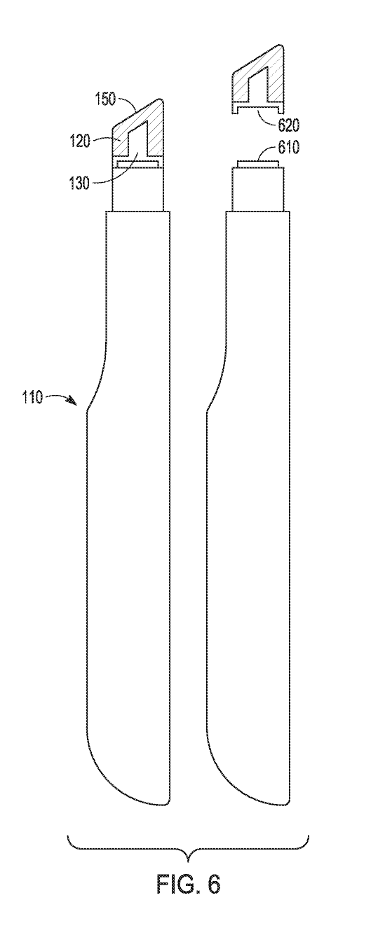

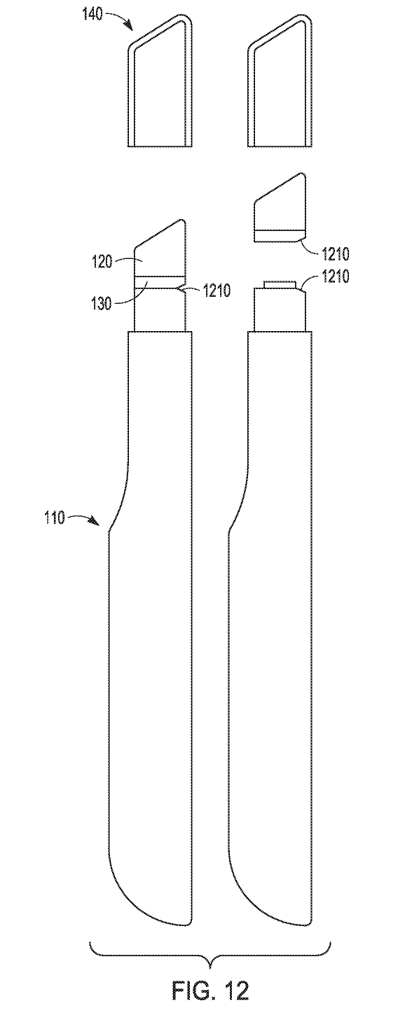

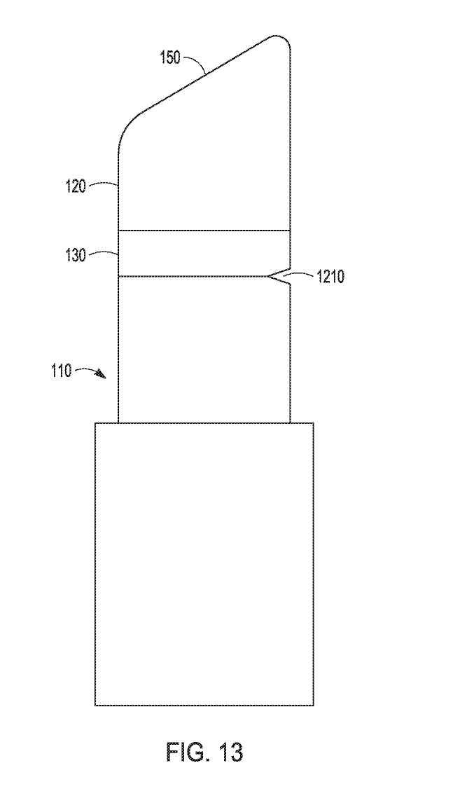

The tip support 130 may connect to the handle 110 through any of various suitable connection mechanisms. In some embodiments, the tip support 130 and handle 110 may connect through a male 610 and female 620 component, allowing the two to snap together, as shown in FIG. 6. For example, the handle 110 may have a male component 610 that extends up from the handle. The component 610 may generally have any shape, and in some embodiments may have a semi-circular or semi-oval shape, similar to the first portion 210 of the handle 110. The component 610 may have a smaller diameter or width than the first portion 210 of the handle 110. The tip support 130 may have a corresponding female component 620 shaped to connect with the male component 610. The female component 620 may be an opening with a shape matching that of the male component 610, which in some embodiments may be a semi-circle or semi-oval shape, similar to that of the first portion 210 of the handle 110. The opening may have a smaller diameter or width than the tip support 130, such that it is surrounded on all sides by the tip support. In some embodiments, the male 610 and female 620 components may be reversed, such that the male component is on the tip support 130 and the corresponding female component 620 is on the handle 110. In other embodiments, the tip support 130 and handle 110 may be threaded, such that the tip support may screw onto the handle, or the tip support 130 may slide onto the handle 110 in a tongue and groove arrangement. Other coupling mechanisms including, but not limited to, friction fit, bayonet connection, adhesives, etc., may additionally or alternatively be used in other embodiments. In some embodiments, the handle 110 and tip support 130 may be formed as a single component. In further embodiments, the whitening tip 120 may connect directly to the handle 110 without a tip support 130. The whitening tip 120 and/or tip support 130 may be removable and/or replaceable, according to some embodiments. The whitening tip 120 and/or tip support 130 may be removable via one of the attachment mechanisms listed above, such as a male/female connection, tongue/groove, or threaded mechanism. Other attachment mechanisms such as those described above may additionally or alternatively be used. In some embodiments, as shown in FIGS. 12 and 13, there may be a cut out or indent 1210 near the whitening tip 120 and/or tip support 130. The cut out 1210 may have a triangular or other shape, cut inward toward the center of the wand 100. The cut out 1210 may be located on the handle 110, whitening tip 120, and/or tip support 130. One embodiment is shown in FIGS. 12 and 13 wherein a cutout 1210 is located on both the handle 110 and the tip support 130. Generally, a tool, such as a fingernail for example, may be inserted into the cut out 1210 to pop the whitening tip 120 and/or tip support 130 off from the handle 110. Generally, any attachment/detachment mechanism may be used.

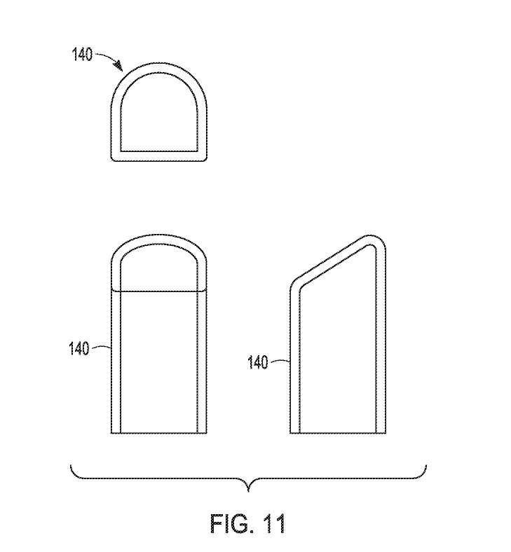

Some embodiments may include a cover, lid, or cap 140, as shown in FIG. 1, and more specifically in FIG. 11. The cap 140 may generally fit, and in some embodiments snugly fit, around or over the whitening tip 120, tip support 130, and/or a portion of the handle 110. The cap 140 may help to protect the whitening tip 120 from bacteria or debris when the wand 100 is not in use. In some embodiments, the cap 140 may be shaped to generally correspond with and surround the whitening tip 120, as shown in FIGS. 1, 2, and 4. In some embodiments the handle 110 may have a lip or ledge 360 that stops the cap 140 and holds it in place when the cap is on the wand 100, as shown in FIGS. 1 and 4. In other embodiments, the cap 140 may attach to the wand 100 with threading, a hinge, or other mechanisms such as, but not limited to, snap fit, friction fit, bayonet connection, etc. The cap 140 may be constructed of rigid plastic, which may be a recyclable plastic such as high or low density polyethylene for example, according to some embodiments.

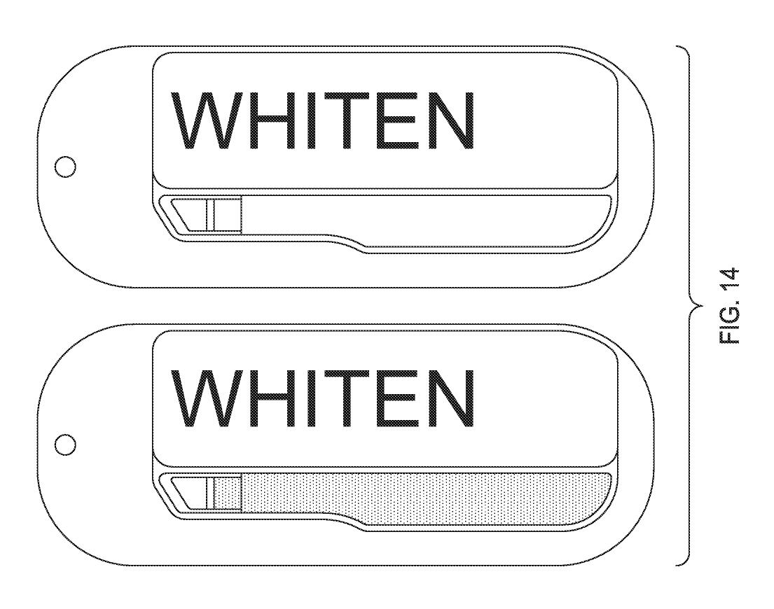

In use, the various embodiments of whitening wands of the present disclosure may assist with tooth whitening and tooth stain removal. A whitening wand may be used with natural teeth, veneers, caps, dentures, or other types of teeth. A user may, according to some embodiments, hold a wand at an angle ranging from 0° to 90°, but more typically from 20° to 45°, from a front surface of his or her teeth, or at an angle such that the angled surface 150 is substantially flat against the user's teeth. The user may then rub or “brush” the teeth using a whitening tip 120. In some embodiments, the user may rub or “brush” with the whitening tip 120 using an up-and-down, side-to-side, or circular motion over the teeth. As the user rubs the whitening tip 120 over his or her teeth, the whitening tip may act to remove or lighten stains on the user's teeth, giving the teeth a whiter and brighter appearance. The whitening tip 120 may absorb stains and/or dental plaque from a tooth's surface. In some embodiments, teeth may appear immediately whiter, and any plaque or stains removed may appear on the whitening tip 120 after use. The whitening tip 120 may be used dry, or a user may moisten the whitening tip before removing stains. According to some embodiments, the whitening wand 100 may be designed and/or packaged for female or male users, as shown in FIG. 14.

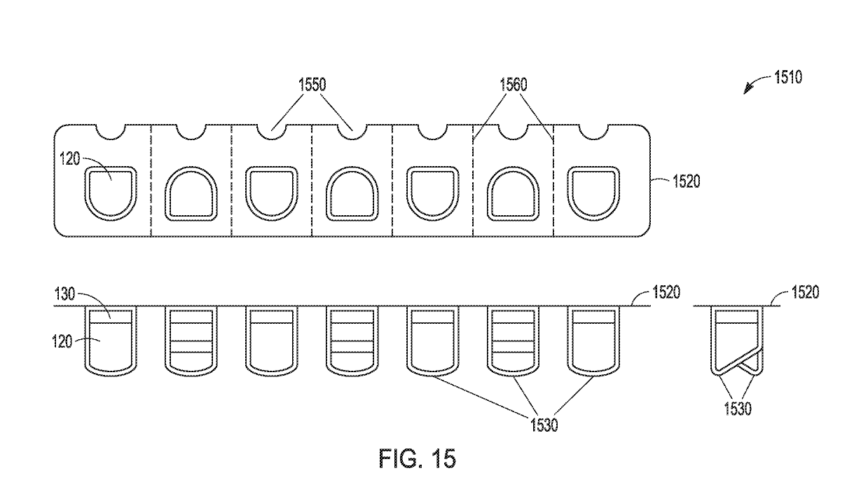

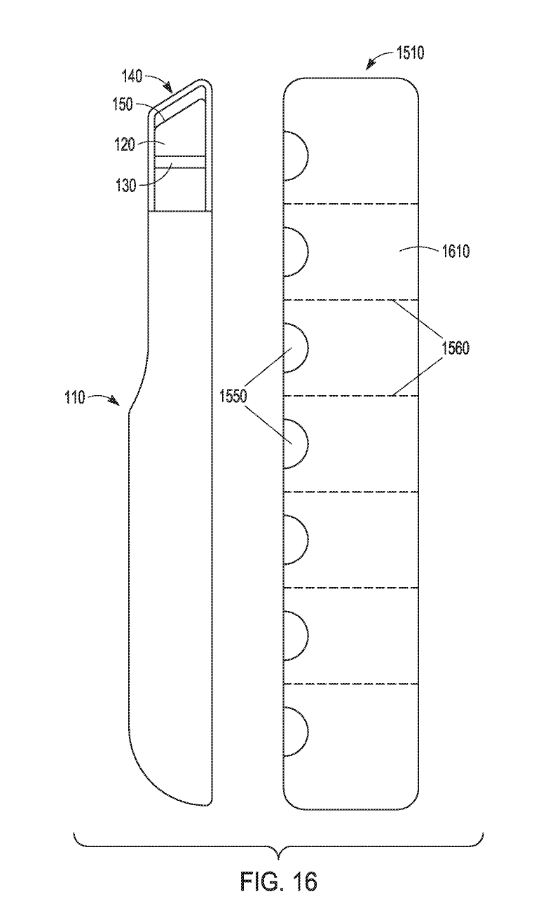

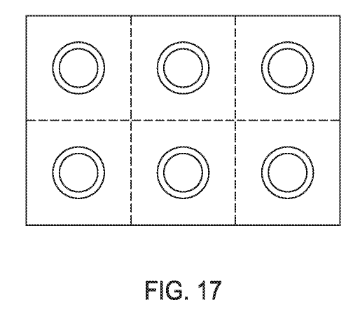

In some embodiments, whitening tips 120 may be designed for limited use. For example, whitening tips 120 may be designed for a single use or to last for multiple uses over a day, week, month, or other period of time. In some cases, the entire whitening wand 100 may similarly be designed for a single use or to last for multiple uses over a day, week, month, or other period of time.

Where the whitening tip 120 is designed for limited use, individual whitening tips and/or tip supports 130 may be packaged separately, such that a user may change the whitening tip and/or tip support when needed. For example, as shown in FIGS. 15 and 16, multiple whitening tips 120 and/or tip supports 130 may be packaged in a blister package 1510, such as that commonly used for pharmaceutical applications. FIG. 17 shows one example of a typical pharmaceutical blister package. In some embodiments, the blister package 1510 may have a plastic wrapper 1520 that forms a pocket or “blister” 1530 for one or more whitening tips 120 and/or tip supports 130. In other embodiments, the wrapper 1520 may be composed of other materials such as paper, cardboard, metal, or other materials. Each blister 1530 may be formed to have the same general shape as the whitening tip 120 or tip support 130 and may be only slightly larger than the tip or tip support, such that the tip or tip support generally fits, and in some cases snugly fits, within the blister. In other embodiments, each blister 1530 may be any size and may have a different shape than that of the tip 120 or tip support 130. In other embodiments, multiple whitening tips 120 and/or tip supports 130 may be packaged in a single blister 1530. The wrapper 1520 may form a different blister 1530 for each whitening tip 120 and/or tip support 130, as shown in FIG. 15. Each of the blisters 1530 may be arranged such that they protrude from the same side of the blister package 1510. The whitening tips 120 and/or tip supports 130 may be packaged in the blisters 1530 such that the side of each tip or tip support that is configured for attachment to the handle 110 is facing the same side of the blister package 1510. The whitening tips 120 and/or tip supports 130 may also be arranged in the blisters 1530 such that the flat side of each tip or support alternates in the direction that it faces, as shown in FIG. 15. This may add stability to the blister package 1510.

At least one side of the blister package 1510, may be covered with a foil sheet or layer 1610. The foil sheet 1610 may be easily peeled away or broken, in some embodiments, such that a user may remove the packaged whitening tip 120 and/or tip support 130. For example, the foil sheet 1610 may be coupled to the wrapper 1520 with a light adhesive, and/or may be thin enough to puncture with a tool such as a fingernail, for example. In some embodiments, the blister package 1510 may be designed such that the tips 120 and/or tip supports 130 may be removed by pushing on the outside of the individual blisters 1530, such that a tip and/or tip support is pushed through the foil sheet 1610 on the opposing side of the package. Further, some embodiments may include one or more cut outs 1550 on the wrapper 1520 and/or foil sheet 1610. The one or more cut outs 1550 may allow a user to more easily grasp and remove the foil sheet 1610, so as to extract a whitening tip 120 and/or tip support 130. In addition, the cutout 1550 of the blister package 1510 may, according to some embodiments, be aligned with the cut out 1210 on or near the whitening tip 120, such that the blister package cut out can be used as a tool and inserted into the cut out near the whitening tip in order to remove the tip and/or tip support 130 from the handle 110. In some embodiments, individual blisters 1530 may be separated by perforations 1560, as shown in FIGS. 15 and 16, such that a user may separate the individual blisters.

In further embodiments, other packaging materials and/or designs may be used for separately packaged whitening tips 120 and/or tip supports 130. For example, in one embodiment, additional tips 120 and/or tip supports 130 may be packaged in a rigid container, such as a container composed of rigid plastic, glass, metal, or other material(s). The rigid container may have a separate compartment for each tip 120 and/or tip support 130. Each compartment may be shaped to generally correspond with and surround the tip 120 and/or tip support 130. As described above with respect to the blister package 1510, each tip 120 and/or tip support 130 may be arranged in the package such that they face the same or different directions. Generally, the side of each tip 120 and/or tip support 130 that couples to the handle 110 may be arranged to face the same direction of the plastic container. The tips 120 and/or tip supports 130 in the plastic container may be accessible via one or more openings. In some embodiments, one side of the plastic container may have a covering that opens to provide access to all of the tips 120 and/or tip supports 130 in the package. The covering may be attached to the container with one or more hinges in some embodiments. In other embodiments, the covering may attach with any of other various mechanisms such as, but not limited to, snap fit, friction fit, bayonet connection, etc. Where the covering attaches to the container with one or more hinges, the covering may have a tab that a user may easily grasp to open the container and access the tips 120 and/or tip supports 130.

Tooth whitening wands may also be formulated to include any one or more of the ingredients identified above or elsewhere herein or combinations thereof, and for achieving similar purposes as described herein with respect to other oral care products. For example, each whitening tip 120 constructed of wood, wood-based material(s), sponge material(s), and/or other suitable materials may be infused or diffused with a material or materials such as, for example, one or more essential oils and/or any of the additional ingredients described herein. For example, various essential oils may have deodorizing properties and thus be used to help prevent or reduce bad breath. Additionally or alternatively, by using one or more essential oils, various flavors or flavor combinations may be provided. In one non-limiting embodiment, potential ingredients used to formulate a tooth whitening wand may include, but are not limited to, essential oils, xylitol, activated charcoal, vitamins, and/or minerals.

The whitening tip 120 may be infused or diffused with one or more materials by various methods such as, but not limited to, submersion or immersion in an essential oil or other material or placement in close proximity, but not in contact, to the material over an extended period of time. In some embodiments, a whitening tip 120 may be such that a user can infuse or diffuse it with an essential oil or other material. For example, the whitening tip 120 and/or tip support 130 may be easily removable by a user for infusion or diffusion by the user, or it may be submersed in an essential oil or other material while still attached to the handle 110. In some embodiments, a whitening wand of the present disclosure may be sold with one or more essential oils or other materials such that a user has all materials needed to infuse or diffuse the whitening tip 120 themselves.

In some embodiments, the one or more essential oils or other materials may be infused or diffused within a whitening tip 120 such that they may be released onto a user's teeth on contact. For example, where the whitening tip 120 is a nano-technology or other sponge or wood-based material having capillaries, the capillaries may hold the essential oils or other materials within the whitening tip, and when the user rubs the whitening tip against his or her teeth, some or all of the oils or other materials may be expelled onto the user's teeth. In other embodiments, the essential oils or other materials may be contained within compartment within the whitening wand 100 or whitening tip 120. The whitening tip 120 may then have one or more channels whereby the oils or other materials may be directed so that they may come in contact with a user's teeth and/or the whitening tip. In some embodiments, a plunger or other mechanism may be used to push the oils or other materials up through the channels. For example, a user may turn, twist, or push a portion of the whitening and 100 or whitening tip 120 in order to direct the oils or other materials through one or more channels. In other embodiments, all or a portion of the whitening wand 100 may be squeezable, such that a user can squeeze the wand to push the oils or other materials onto or into the whitening tip 120.

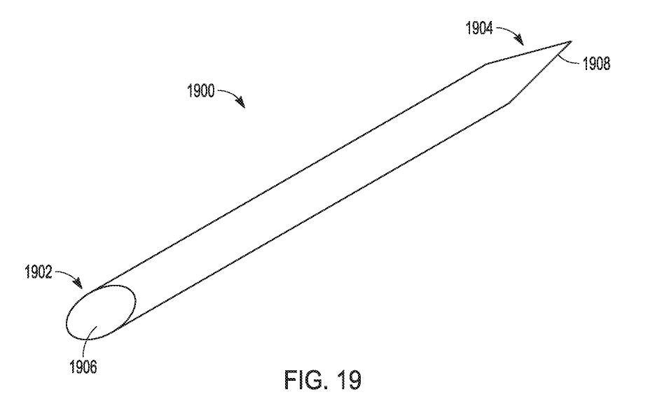

FIG. 19 shows another embodiment of a whitening wand 1900 according to one or more embodiments. In some embodiments, the wand 1900 may be constructed entirely or primarily of wood or wood-based components. For example, the wand 1900 may generally be constructed from a wooden dowel having a cylindrical shape. As shown, the wand 1900 may generally have an elongated shape extending between a first end 1902 and a second end 1904. The wand 1900 may have a length of between approximately 2 inches and approximately 8 inches in some embodiments. In other embodiments, the wand 1900 may have any other suitable length. The wand 1900 may have a rounded cross sectional shape as shown in FIG. 19. However, in other embodiments, the wand 1900 may have any other suitable cross sectional shape. In some embodiments, the wand may have a generally consistent width or diameter across its length, as shown in FIG. 19. In some embodiments, the wand 1900 may have padding and/or contouring configured to accommodate or fit to a user's hand such that a user may comfortably hold the wand.

At the first end 1902, the wand may have a whitening tip 1906 with one or more angled surfaces configured to be rubbed, scraped, or brushed against a user's teeth. The whitening tip 1906 may be angled similarly to the whitening tips 120 described above. In some embodiments, the second end 1904 may have a pointed tip 1908, where the cross section of the wand at the second end may narrow to a relatively sharp or dull point. The pointed tip 1908 may also be configured for rubbing, scraping, or brushing against a user's teeth, which may allow a user to address otherwise hard to reach areas of her or his teeth. In some embodiments, the wand 1900 or portions thereof may be infused or diffused with essential oils and/or other ingredients described herein, similar to the whitening tips described above. Moreover, where the entire wand 1900 is constructed of wood or wood-based products, in some embodiments, the entire wand may be infused or diffused with the essential oil(s) and/or other ingredients.

In some embodiments, any of the whitening wands or whitening tips of the present disclosure may be disposable and/or replaceable. Wands or whitening tips may be designed for single use or for multiple uses over a day, week, month, or other period of time. The whitening tip may be infused with one or more essential oils or other materials, according to some embodiments. One or more essential oils may be chosen for its whitening, antimicrobial, antiseptic, and/or antibacterial benefits. Additionally, one or more essential oils may be chosen for flavoring or perfumery, or for desired mental or physical effects on the user.

Oral Care Crystals

In accordance with the present disclosure, one or more unique oral care crystals may be created including any one or more of the ingredients identified above or elsewhere herein or combinations thereof, and for achieving similar purposes as described herein with respect to other oral care products. For example, oral care crystals may be or include xylitol crystals. In some embodiments, xylitol crystals may be used to freshen one's breath after a meal, or at any other desired time. Such crystals may be tailored for specific users, times, or circumstances. For example, a xylitol crystal may be designed for use post lunch. In at least one embodiment, potential ingredients for an after-lunch xylitol crystal formula may include, but are not limited to, potassium bicarbonate, vitamin B12, chromium, and/or one or more essential oils or other ingredients. In one embodiment, the crystals may, additionally or alternatively, include a proprietary blend breath freshener. Additionally, xylitol crystals or other oral care crystals may help to destroy bacteria that may otherwise lead to plaque and decay. Oral care crystals may include one or more essential oils and/or other ingredients to help penetrate and destroy biofilm as well. Oral care crystals may additionally or alternatively be formulated to mitigate dry mouth and/or to whiten teeth.

In some embodiments, xylitol crystals or other oral care crystals may be configured to be arranged in a dental tray, such that the tray may hold the crystals against or around a user's teeth and/or gums. The dental tray may be any suitable dental tray, such as a night guard, configured to fit over or across the user's teeth. Such a dental tray may be constructed of one or more plastics and/or any other suitable materials. The dental tray may be molded or shaped for a particular user's teeth. In some embodiments, oral care crystals may be arranged or formed on a strip for placing within a dental tray or for placing directly across a user's teeth. Dental trays and/or strips may be used to hold the oral care crystals against or proximate to a user's teeth and/or gums for a desired amount of time. In some embodiments, the oral care crystals may be configured to dissolve.

As may be appreciated, refreshing mints, hard candies, and/or gum may also be created incorporating any one or more of the ingredients identified above or elsewhere herein.

Dental Brush

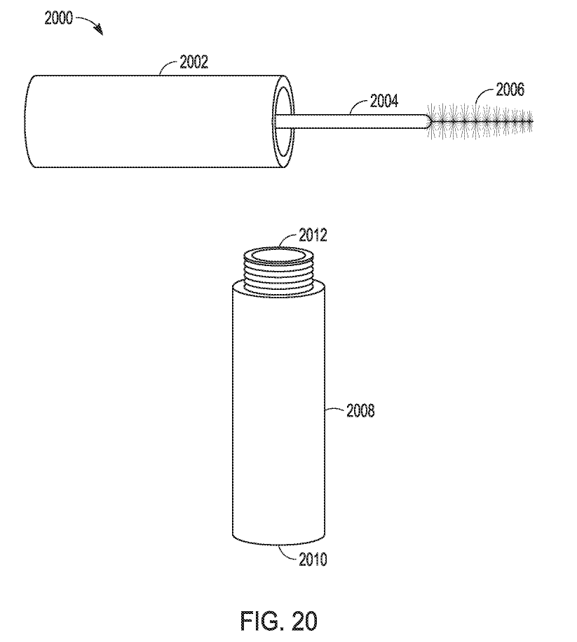

In accordance with the present disclosure, one or more unique dental brush products may be created including any one or more of the ingredients identified above or elsewhere herein or combinations thereof, and for achieving similar purposes as described herein with respect to other oral care products. FIG. 20 shows one embodiment of a dental brush 2000 of the present disclosure. The dental brush 2000 may include a handle portion 2002, a wand portion 2004, and a brush portion 2006.

The handle portion 2002 may be configured such that a user may grip or hold the handle with one hand while using the brush on the user's teeth. In some embodiments, the handle portion 2002 may have contouring to fit a user's hand comfortably. The wand portion 2004 may extend from the handle portion 2002. Each of the wand 2004 and handle 2002 portions may have any suitable length and width. For example, each of the wand portion 2004 and handle portion 2002 may have a length of between approximately two inches and approximately 5 inches. The handle portion 2002 may have a width or diameter suitable for a user to comfortably grasp the handle portion with one hand

The brush portion 2006 may extend from, or be arranged on, the wand portion 2004. The brush portion 2006 may include a plurality of brush fibers, which may be plastic fibers for example, configured for brushing or scrubbing a user's teeth and/or gums. In some embodiments, the brush fibers may be arranged in a spiral shape or in a plurality circular shapes centered around a central shaft, as shown in FIG. 20. In this way, brush fibers may extend from all sides of the brush portion 2006. In other embodiments, the brush portion 2006 may be configured such that brush fibers extend from one side or two sides of the brush, for example. Moreover, the brush fibers may be arranged in one or more lines or rows, in one or more clusters, in a rectangular or square shape, and/or in any other suitable shape on the brush portion 2006. The brush fibers may have any suitable length configured to brush or scrub a user's teeth and/or gums. The brush portion 2006 may have any suitable length and width configured to accommodate the brush fibers.

In some embodiments, the dental brush 2000 may include a case portion 2008. The case portion 2008 may be an enclosure configured to receive the brush portion 2006 and/or wand portion 2004 of the dental brush. For example, as shown in FIG. 20, in some embodiments, the case portion 1908 may have a tubular shape extending between a closed end 2010 and an open end 2012. The dental brush 2000 may be configured such that the brush portion 2006 and wand portion 2004 may be passed through the open end 2012 so as to be arranged within the case portion 2008. In some embodiments, the wand portion 2004 and/or handle portion 2002 may be configured to couple to the case portion 2008. For example, the handle portion 2002 and case portion 2008 may have threading configured for engagement to thread the handle portion onto the case portion, thus closing or sealing the open end of the case portion with the wand portion and brush portion inside.

While the case portion 2008 may provide a carrying case for the dental brush, it may additionally allow a user to apply toothpaste or another product to the brush portion 2006. For example, a toothpaste or another paste, gel, or liquid may be arranged within the case portion 2008, such that each time a user inserts the brush portion 2006 into the case portion, the brush portion may contact and become at least partially coated with the toothpaste or other formulation. In some embodiments, the toothpaste, paste, gel, or liquid may be provided in the form of a capsule, similar to those described above with respect to dental floss. The toothpaste, other paste, gel, or liquid may include any combination of the ingredients discussed above. In particular, any of the toothpaste formulations discussed above may be include in the case portion 2008.

In some embodiments, the toothpaste, other paste, liquid, or gel may include one or more ingredients that give the dental brush a unique flavor and/or provide one or more health benefits. For example, a dental brush designed for evening use may be infused or diffused with lavender and/or chamomile. In another embodiment, a dental brush designed for morning use may be infused or diffused with citrus and/or mint essential oils. In various embodiments, a dental brush of the present disclosure may additionally include one or more essential oil infusions that strengthen gums, such as cypress, lemon, and/or peppermint. As may be appreciated, dental floss of the present disclosure may be infused or diffused with any suitable essential oil, natural botanical, and/or other suitable ingredient.

Cleaners/Whiteners

In accordance with the present disclosure, one or more unique tooth cleaning products may be created including any one or more of the ingredients identified above or elsewhere herein or combinations thereof, and for achieving similar purposes as described herein with respect to other oral care products. Teeth are subject to staining, microorganism growth, and odor.

Mixing

Formulations of the present disclosure, including water, oils, essential oils, solids, and/or other components, may be combined or mixed using any suitable methods or processes. In some embodiments, a high speed centrifugal force or high sheer homogenization process may be applied to the two or more formulating ingredients in order to create a mixture. For example, a nanoemulsion high sheer process may be used to disperse a phase (such as an oil or oil-based component, powder or other solid, or another component) into a main continuous phase (such as an aqueous component) in which the phase would not typically be considered miscible. Such a high sheer process may create nanoparticles of the phase that are less than approximately 100 nanometers in size, allowing for an equilibrium dispersion of particles within the main phase. More specifically, a high speed centrifugal force or mixing may be applied when the formulation comprise one or more water-based solids or liquids and one or more oil-based solids or liquids (such as one or more essential oils), in order to cause the ingredients to bind, become miscible, or form a generally stable and uniform suspension or mixture. That is, the high speed centrifugal force or mixing may generally facilitate the emulsion of the oils with water, and/or one or more other ingredients.

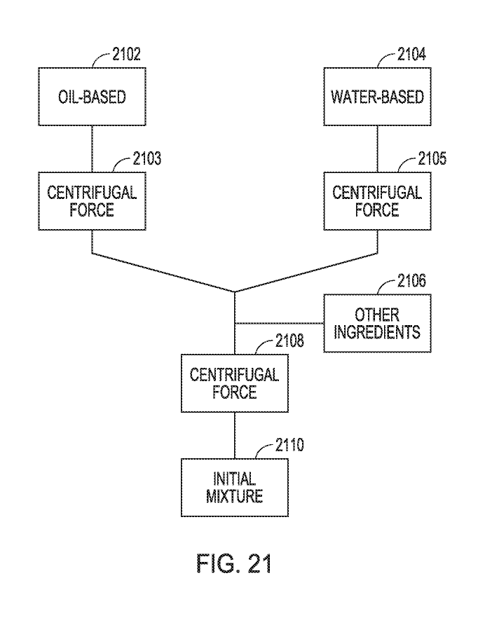

For example and example only, adding one or more ingredients, such as an essential oil and water, to a blender or mixing device and exposing the ingredients to the centrifugal or homogenizing mixing forces of the blender or mixing device for a specified period of time, can bind, mix, suspend, or otherwise emulsify the one or more ingredients. Referring to FIG. 21, a method 2100 to combine one or more oil-based solids or liquids 2102 with one or more water-based solids or liquids 2104 may be seen. In various embodiments, one or more oil-based solids or liquids 2102 and one or more water-based solids or liquids 2104 may be combined using centrifugal force or other high speed mixing 2108 to create a formulation or initial mixture 2110. In some embodiments, some or all of the one or more oil-based solids or liquids 2102 may be subject to a high sheer, centrifugal force, or homogenizing force 2103 prior to their combination with the water-based solids or liquids 2104. For example, multiple essential oils may be mixed together using a high sheer mixing process in order to ensure a more homogenous oil mixture prior to being combined with one or more water-based materials. Similarly, in some embodiments, some or all of the one or more water-based solids or liquids 2104 may be subject to a high sheer, centrifugal force, or homogenizing force 2105 prior to their combination with the oil-based solids or liquids 2102.

The one or more oil-based products 2102 and one or more water-based products 2104 may be combined and subject to the centrifugal or homogenizing force 2108. In some embodiments, one or more additional ingredients 2106 may, additionally or alternatively, be added to the water-based material(a) 2104 and oil-based material(s) 2102 prior to, during, or after application of the centrifugal or homogenizing force 2108. For example, an emulsifier may be used to further assist in keeping the two or more ingredients from separating. However, in various embodiments of the present disclosure, an emulsifier, or other ingredient 2106, may be unnecessary in the creation of a substantially homogenous or substantially uniform mixture 2110 using the centrifugal or homogenizing force 2108. In some embodiments, formulations made with such ingredients and mixed in this manner may stay mixed together for substantially extended periods of time, and in some cases even stay permanently mixed. Thus, after application of the centrifugal or homogenizing force 2108, the initial mixture 2110 may be stored for an extended period of time without the need for additional mixing. It is to be appreciated that the reduction of particle size of essential oils and/or other components of the formulations of the present disclosure produces produced by such nanoemulsion processes may lead to better absorption and penetration of the formulations into the teeth, gums, and/or other surfaces.

Other Contemplations

As discussed above, the various toothpastes, mouthwashes, whitening products, effervescent crystals, dental floss, and other products may be formulated to be uniquely adapted to one or more users or experiences. While some examples were given in the discussion of each product, they are not intended to be limiting. The current disclosure includes a line of oral care products that may be adapted or formulated for a variety of different users or experiences. Therefore, all the products herein are capable of being formulated for AM use or PM use. Other uses are contemplated, some of which are described below.

In various embodiments, products may be formulated to coincide with certain meals. For example, many people dislike the taste of coffee just after brushing their teeth in the morning. However, more and more people prepare a cup of coffee to take with them to work or grab one on-the-go. In various embodiments, oral care products, such as toothpaste, may be formulated to include a coffee essential oil. In other embodiments, another essential oil or ingredient having either a less minty flavor and/or a bitter flavor may make for a more desirable transition to drinking coffee after brushing one's teeth. Similarly, a person who dislikes the taste of drinking orange juice after brushing their teeth in the morning may wish to use toothpaste and/or a rinse with a citrus taste appeal. In some embodiments, the toothpaste may include essential oils or natural botanicals having a citrus taste, including but not limited to, orange, lemon, lime, mandarin, and tangerine. Of course, other flavors are contemplated for other meals or times of day. For example, in one embodiment, a toothpaste may include a vanilla and/or chocolate essential oils for use before bed. This may encourage users to brush their teeth as it may taste like dessert after a meal.

In various embodiments, products may be formulated to combat or aid in fighting different ailments or illnesses. For example, vitamin C is commonly used to prevent or treat the common cold. Oral care products contemplated herein may include vitamin C, or increased levels of vitamin C, for consumers during cold and flu season or while infected. In another example, consumers with osteoporosis may desire oral care products with increased calcium. Vitamin D, vitamin C, vitamin E, vitamin K, and magnesium have been shown to assist in calcium absorption. Thus, in some embodiments, oral care products herein may be formulated to include calcium, and may additionally include vitamin D, vitamin C, vitamin E, vitamin K, and/or magnesium. Other ailments or illnesses may similarly be combatted by adding one or more ingredients known or thought to help fight or prevent such ailment or illness to the various oral care products of the present disclosure.

In various embodiments, products may be formulated for varying age ranges. Most people have more intense taste sensations early in life and lose some taste sensations as they age. Therefore, in some embodiments designed for children, the flavor profile of the products may be light and sweet. In other embodiments designed for senior citizens, the flavor profile of products may be intense and/or bitter.

In various embodiments, products may be formulated for the time of year, e.g., seasons or holidays. In some embodiments designed for autumn or fall, essential oils in the products may include, but are not limited to, allspice, cardamom, cinnamon, clove, coffee, ginger, nutmeg, sage, and/or rosemary. In other embodiments designed for summer, essential oils in the products may include, but are not limited to, citronella, basil, fennel, grapefruit, jasmine, juniper berry, lavender, lemon, lemongrass, lime, orange, rose, rosemary, spearmint, thyme, violet, yuzu, and/or cypress. In another embodiment designed for spring, essential oils in the products may include, but are not limited to, tea tree, rose, parsley, basil, dill, mandarin, lemon, eucalyptus, and/or tangerine. In still another embodiment designed for winter, essential oil ingredients may include, allspice, anise, holy basil, Peru balsam, bay, black pepper, chocolate, peppermint, cinnamon, clove, coffee, sage, juniper berry, nutmeg, fir needle, frankincense, oakmoss, scotch pine, spruce, sweet orange, and/or vanilla.

In various embodiments, products may be custom formulated by the user. That is, a user may be able to customize the ingredients in each product in order to obtain their desired results: cleaning, health benefit, and/or flavor. In some embodiments, each product (toothpaste, mouthwash, etc.) may be created using a base formulation. A user may then be able to add one or more essential oils to the formula to obtain the desired results. In one embodiment, the user may be able to custom make his or her oral care product via a website and have the product later shipped to them. In another embodiment, the user may be able to purchase the base formula and add one or more essential oils at home. Any suitable method to create custom made oral care products in accordance with the present disclosure may be used.

In some embodiments, products of the present disclosure may be formulated without the use of particular ingredients. For example, a toothpaste or other product may be provided without sodium laurel sulfate. Sodium laurel sulfate is a foaming agent that may increase the frequency and/or intensity of canker sore outbreaks and may be toxic. A toothpaste or other product may be provided without triclosan. Triclosan is used to kill bacteria, but may be toxic, may disrupt hormones, and may be environmentally damaging. A toothpaste or other product of the present disclosure may be provided without artificial colors or flavors. In general, products of the present disclosure may include natural and non-GMO ingredients, and may avoid the use of artificially produced chemicals and components. A toothpaste or other product of the present disclosure may be provided without hydrated silica. Hydrated silica is a stain remover that may build up in or on teeth and may lead to damage to the tooth enamel and/or dulling of the teeth. Additionally, a toothpaste or other product of the present disclosure may be provided without alcohol. Alcohol is used to kill bacteria, but may dry gums, damage soft tissue, and lead to inflammation.

Some products of the present disclosure may be provided or packaged in biophotonic glass containers, or other biophotonic containers. Biophotonic containers or packaging may by opaque or partially opaque and may be configured to filter out at least some light from entering the container. For example, biophotonic glass may filter visible sunlight, so as to allow infra-red and ultraviolet light to penetrate the container. This may help to preserve some products and may help increase or maintain stability of the products in some embodiments.

It is to be appreciated that formulations described herein may be helpful in removing or preventing the formation of biofilm in the oral cavity. Dental biofilm is a sticky substance that forms on and adheres to teeth. If left untreated, biofilm may lead to gingivitis and periodontal disease. Further, small microscopic particles of this dental biofilm may “break off” and be absorbed by the very porous surface of the oral mucosa. Once absorbed, the bacteria laden biofilm particle may pass into the bloodstream and subsequently find its way to body organs and tissues. At this point, because of its sticky nature, the biofilm can easily adhere to any surface, such as the inner wall of the heart, the interior wall of a blood vessel, or even a joint area. Once established, these bacterial colonies can be difficult to treat even with the use of antibiotics. Essential oils may be beneficial and effective in treating, removing, and/or preventing dental biofilm. In particular, essential oils can penetrate the deep areas of the oral cavity, as well as provide antibacterial, antifungal, and antiviral activity in the deep recesses of the gum line. Essential oils can also provide nourishment to the dentin and root system of teeth and provide an anti-inflammatory benefit.

Additionally, formulations described herein may be helpful in inhibiting matrix metalloproteinases (MMPs) in the oral cavity. MMPs are enzymes that break down proteins in the body, including in tooth material. For example, when a tooth is damaged by the formation of a cavity, the body releases and activates MMPs to break down and remove the damaged tooth material. However, there are circumstances where the body may have an excess of, or imbalance of, MMPs, such that the MMPs may break down or attach healthy tissue. For example, a pH within the oral cavity of more or less than 7 may cause excess or undesired activation of MMPs. The presence of free radicals, which may be caused by smoking, pollutants and pesticides, ionizing radiation, stress, nutritional deficiencies, alcohol, or other factors, may cause excess or undesired activation of MMPs. However, essential oils may help to reduce or inhibit MMPs. In this way, oral care product formulations containing essential oils, as described herein, may help to reduce or inhibit excess or undesired MMPs within the body or within the oral cavity, thus helping to protect healthy tissue.

Moreover, formulations of the present disclosure may help to maintain a neutral pH (i.e., a pH of about 7) within the oral cavity. An acidic (pH of less than 7) or alkaline (pH of more than 7) environment within the oral cavity can be harmful. Various internal and external factors can lead to acidic or alkaline conditions in the oral cavity. For example, acids produced by bacteria within the oral cavity can affect pH within the mouth. Particular foods and drinks can affect the pH balance within the oral cavity. Moreover, some conventional toothpastes and mouthwashes can be particularly acidic (with a pH as low as 3.1 in some cases) or particularly alkaline (with a pH as high as 9.8 in some cases), such that their use can lead to an acidic or alkaline pH within the oral cavity. Product formulations described herein may be formulated to have a pH of between about 6 and about 8, between about 6.5 and about 7.5, or between about 6.7 and about 7.3. In some embodiments, product formulations described herein may have a pH of 7 or of approximately 7. In this way, formulations of the present disclosure may help to provide a balanced or neutral pH within the oral cavity. A neutral pH may help promote remineralization of teeth, as well as prevent or inhibit harmful effects associated with an acidic or alkaline oral cavity.

As used herein and in the claims, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an ingredient or element may still actually contain such item as long as there is generally no measurable effect thereof

Additionally, as used herein, the phrase “at least one of [X] and [Y],” where X and Y are different components that may be included in an embodiment of the present disclosure, means that the embodiment could include component X without component Y, the embodiment could include the component Y without component X, or the embodiment could include both components X and Y. Similarly, when used with respect to three or more components, such as “at least one of [X], [Y], and [Z],” the phrase means that the embodiment could include any one of the three or more components, any combination or sub-combination of any of the components, or all of the components.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principals of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

We claim:

1. A toothpaste comprising purified water, xylitol, calcium citrate, and at least one essential oil.

2. The toothpaste of claim 1, further comprising at least one of diatomaceous earth, malic acid, xanthan gum, arrowroot flour, and coconut oil.

3. The toothpaste of claim 2, further comprising potassium sorbate, at least one vitamin, and at least one mineral.

4. The toothpaste of claim 1, wherein the at least one essential oil comprises at least one of: agar, ajwain, allspice, ambrette seed, amyris, angelica root, anise, arnica, asafetida, balsam of Peru, basil, holy basil, bay, bay laurel, beeswax, benzoin, bergamot, blue cypress, bois-de-rose, boronia, black pepper, buchu oil, birch, bitter almond, boldo, cajuput, camphor, brown camphor, yellow camphor, cannabis flower, caraway, cardamom seed, zingiberaceae, carrot seed, cedarwood, chamomile, chocolate (cacao), calamus root, cassia, cinnamon, citronella, clary sage, clove, clover, coffee, coriander, costmary, costus root, cumin, curry leaf, cypress, davana, deertongue, dill, elecampane, elemi, eucalyptus, fennel seed, fenugreek, fir, frankincense, galangal, galbanum, garlic, geranium, ginger, goldenrod, grapefruit, henna, helichrysum, hickory nut, horseradish, hyssop, Idaho tansy, jaborandi, jasmine, juniper berry, laurus nobilis, lavandin, lavender, ledum, lemon, lemon myrtle, lemongrass, lime, litsea cubeba, linaloe, mandarin, manuka, marjoram, melaleuca alternifolia, melilotus, Melissa oil, menthe arvensis, mint oil, mountain savory, mugwort, mullein, mustard, myrrh, myrtle, neem, neroli, niaouli, nutmeg, oakmoss, onion, sweet orange, bitter orange, oregano, orange, orris, palmarosa, Palo Santo, parsley, patchaouli, peppermint, perilla, pennyroyal, petitgrain, pimento, pine, ravensara, red cedar, roman chamomile, rose, rosehip, rosemary, rosewood, rue, sage, sandalwood, sassafras, savory, schisandra, Spanish broom, spearmint, spikenard, spruce, star anise, sweet birch, tagetes, tangerine, tansy, tarragon, tea tree, thuja, thyme, tobacco, tsuga, tuberose, turmeric, valerian, vanilla, violet leaf, Virginian, vetiver, western red cedar, wintergreen, wormseed, wormwood, yarrow, ylang-ylang, zedoary, and yuzu oil.

5. The toothpaste of claim 4, wherein the at least one essential oil comprises a blend of two or more of: lemon oil, lime oil, sweet orange oil, ginger oil, tea tree oil, wintergreen oil, spearmint oil, peppermint oil, ylang ylang oil, vanilla oil, cinnamon oil, clove oil, grapefruit oil, eucalyptus oil and myrrh oil.

6. The toothpaste of claim 1, wherein at least two components of the toothpaste are combined using a homogenization process.

7. The toothpaste of claim 1, wherein the toothpaste comprises between about 20% and about 35% by volume of xylitol, and wherein the xylitol is provided as Birch Tree xylitol.

8. The toothpaste of claim 1, wherein the toothpaste comprises between about 20% and about 35% by volume of xylitol, between about 3% and about 15% by volume of calcium citrate, between about 0.01% and about 4% by volume of the at least one essential oil.

9. The toothpaste of claim 1, wherein the toothpaste comprises a pH of between 6.7 and 7.3.

10. An alcohol-free mouthwash comprising purified water, xylitol, and at least one essential oil.

11. The mouthwash of claim 10, further comprising potassium sorbate, at least one vitamin, and at least one mineral.

12. The mouthwash of claim 10, wherein the at least one essential oil comprises at least one of: agar, ajwain, allspice, ambrette seed, amyris, angelica root, anise, arnica, asafetida, balsam of Peru, basil, holy basil, bay, bay laurel, beeswax, benzoin, bergamot, blue cypress, bois-de-rose, boronia, black pepper, buchu oil, birch, bitter almond, boldo, cajuput, camphor, brown camphor, yellow camphor, cannabis flower, caraway, cardamom seed, zingiberaceae, carrot seed, cedarwood, chamomile, chocolate (cacao), calamus root, cassia, cinnamon, citronella, clary sage, clove, clover, coffee, coriander, costmary, costus root, cumin, curry leaf, cypress, davana, deertongue, dill, elecampane, elemi, eucalyptus, fennel seed, fenugreek, fir, frankincense, galangal, galbanum, garlic, geranium, ginger, goldenrod, grapefruit, henna, helichrysum, hickory nut, horseradish, hyssop, Idaho tansy, jaborandi, jasmine, juniper berry, laurus nobilis, lavandin, lavender, ledum, lemon, lemon myrtle, lemongrass, lime, litsea cubeba, linaloe, mandarin, manuka, marjoram, melaleuca alternifolia, melilotus, Melissa oil, menthe arvensis, mint oil, mountain savory, mugwort, mullein, mustard, myrrh, myrtle, neem, neroli, niaouli, nutmeg, oakmoss, onion, sweet orange, bitter orange, oregano, orange, orris, palmarosa, Palo Santo, parsley, patchaouli, peppermint, perilla, pennyroyal, petitgrain, pimento, pine, ravensara, red cedar, roman chamomile, rose, rosehip, rosemary, rosewood, rue, sage, sandalwood, sassafras, savory, schisandra, Spanish broom, spearmint, spikenard, spruce, star anise, sweet birch, tagetes, tangerine, tansy, tarragon, tea tree, thuja, thyme, tobacco, tsuga, tuberose, turmeric, valerian, vanilla, violet leaf, Virginian, vetiver, western red cedar, wintergreen, wormseed, wormwood, yarrow, ylang-ylang, zedoary, and yuzu oil.

13. The mouthwash of claim 10, wherein the at least one essential oil comprises a blend of two or more of: lemon oil, lime oil, sweet orange oil, wintergreen oil, spearmint oil, peppermint oil, ylang ylang oil, vanilla oil, cinnamon oil, clove oil, grapefruit oil, eucalyptus oil, and myrrh oil.

14. The mouthwash of claim 10, wherein at least two components of the mouthwash are combined using a homogenization process.

15. The mouthwash of claim 10, wherein the mouthwash comprises between about 7% and about 30% by volume of xylitol and between about 0.2% and about 3.5% by volume of the at least one essential oil.

16. The mouthwash of claim 10, wherein the mouthwash comprises a pH of between 6.7 and 7.3.

17. A whitening wand for removing or lightening stains and/or plaque from teeth, comprising:
a handle; and
a porous whitening tip coupled to the handle.

18. The whitening wand of claim 17, wherein the whitening tip comprises a micro-compressed nano-technology sponge.

19. The whitening wand of claim 17, wherein the whitening tip comprises a wood or wood-based component.

20. The whitening wand of claim 17, wherein the whitening tip is readily removable from the handle.
